(19) United States
(12) Patent Application Publication
(10) Pub. No.: US 2006/0275215 A1
(43) Pub. Date: Dec. 7, 2006

(54) Novel imaging agents

(76) Inventors: Duncan Hiscock, Buckinghamshire (GB); Ben Newton, Buckinghamshire (GB); Benedicte Guilbert, Buckinghamshire (GB)

Correspondence Address: Amersham Health Inc;IP Department, 101 Carnegie Center, Princeton, NJ, 08540, US

(21) Appl. No.: 10/560,509

(22) PCT Filed: Nov. 26, 2004
(86) PCT No.: PCT/GB04/05003

§ 371 Date: Aug. 29, 2006

(30) Foreign Application Priority Data
Nov. 26, 2003 (GB) 0327494.1

(51) Int. Cl. A61K 51/00; C12N 9/99; A61K 49/10; A61K 49/00

(52) U.S. Cl. 424/1.69; 424/9.34; 424/9.6; 435/184

(57) ABSTRACT

The present invention relates to diagnostic imaging agents for in vivo imaging. The imaging agents comprise a synthetic caspase-3 inhibitor labelled with an imaging moiety suitable for diagnostic imaging in vivo. The invention also provides pharmaceutical and radiopharmaceutical compositions comprising the imaging agents, together with kits for the preparation of the radiopharmaceuticals. Also described are chelator conjugates of the caspase-3 inhibitor, which are suitable for the preparation of imaging agents comprising a radioactive or paramagnetic metal ion. The imaging agents are useful for the diagnostic imaging and or therapy monitoring in vivo of various disease states where caspase-3 is involved.

### FIELD OF THE INVENTION

The present invention relates to diagnostic imaging agents for in vivo imaging. The imaging agents comprise a synthetic caspase-3 inhibitor labelled with an imaging moiety suitable for diagnostic imaging in vivo.

### BACKGROUND TO THE INVENTION

Programmed cell death by apoptosis is a complex process, involving a large number of cellular processes with numerous levels of control. It is initiated by one of two pathways. The first is through an extrinsic pathway initiated via a cell surface death receptors and the second is through intrinsic initiators, such as DNA damage by UV radiation. Both of these pathways culminate in the co-ordinated death of cells which requires energy and, unlike cell death by necrosis, does not involve an inflammatory response. Cells committed to apoptosis present ‘eat me’ signals on their cell surface, which invite other cells to consume them by phagocytosis.

Apoptosis is a critical event in numerous processes within the body. For example, embryonic development is totally reliant on apoptosis, and tissues that turnover rapidly require tight regulation to avoid serious pathological consequences. Failure to regulate apoptosis can give rise to cancers (insufficient cell death) and neuropathologies such as Alzheimer's disease (too much cell death). Furthermore, apoptosis can also be indicative of damaged tissues such as areas within the heart following ischaemia/reperfusion insults.

Annexin-5 is an endogenous human protein (RMM 36 kDa) which binds to the phosphatidylserine (PS) on the outer membrane of apoptotic cells with an affinity of around 10⁻⁹ M. ^{99m}Tc-labelled Annexin-5 has been used to image apoptosis in vivo [Blankenberg et al, J. Nucl. Med., 40, 184-191 (1999)]. There are, however, several problems with this approach. First, Annexin-5 can also enter necrotic cells to bind PS exposed on the inner leaflet of the cell membrane, which could lead to false-positive results. Second is the high blood pool activity, which is maintained for at least two hours after injection of labelled annexin-5. This means that the optimal timing of imaging is between 10 and 15 h after injection [Reutelingsperger et al, J. Immunol. Meth., 265 (1-2), 123-32 (2002)], making it unsuitable for clinical decision making in patients with acute coronary syndromes. Furthermore, the clearance of annexin-5 occurs via the kidney and the liver, with a very strong background signal in the abdominal regions. This makes imaging of abdominal cell death (eg. in kidney transplants and tumour monitoring) impossible.

WO 01/89584 discloses at Examples 16 to 18 and 21 that a chelator conjugate of the caspase-3 substrate tetrapeptide DEVD (ie. Asp-Glu-Val-Asp) may be useful for in vivo imaging of apoptopic tissue using MRI or scintigraphy.

Haberkorn et al [Nucl. Med. Biol., 28, 793-798 (2001)] studied the pan-caspase inhibitor, Z-VAD-fmk ie. benzyloxycarbonyl-Val-Ala-DL-Asp(O-methyl)-fluoromethylketone labelled with the radioisotope ¹³¹I as a potential apoptosis imaging agent. They found the absolute cellular uptake of the agent to be low, and attributed this to the trapping of only one inhibitor molecule per activated caspase. They concluded that a labelled caspase substrate should not suffer from this problem and would be a better approach for an imaging agent.

Radiopharmaceuticals for apoptosis imaging have been reviewed by Lahorte et al [Eur. J. Nucl. Med., 31, 887-919 (2004)].

There is therefore still a need for an apoptosis imaging agent which permits rapid imaging (eg. within one hour of injection), and with good clearance from blood and background organs.

### THE PRESENT INVENTION

It has now been found that synthetic caspase-3 inhibitors labelled with an imaging moiety are useful diagnostic imaging agents for in vivo imaging of those diseases of the mammalian body where abnormal apoptosis, especially where excessive apoptosis is involved.

The imaging moiety can be radioactive (eg. a radioactive metal ion, a gamma-emitting radioactive halogen or a positron-emitting radioactive non-metal) or non-radioactive (eg. a paramagnetic metal ion, a hyperpolarised NMR-active nucleus or an optical dye suitable for in vivo imaging).

Excessive apoptosis is associated with a wide range of human diseases, and the importance of caspases in the progression of many of these disorders has been demonstrated. Hence, the imaging agents of the present invention are useful for the in vivo diagnostic imaging and or therapy monitoring in a range of disease states, which include:
(a) acute disorders, such as response to cardiac and cerebral ischaemia/reperfusion injury (eg. myocardial infarction or stroke respectively), spinal cord injury, traumatic brain injury, organ rejection during transplantation, liver degeneration (eg. hepatitis), sepsis and bacterial meningitis;
(b) chronic disorders such as neurodegenerative diseases (eg. Alzheimer's disease, Huntington's Disease, Down's Syndrome, spinal muscular atrophy, multiple sclerosis, Parkinson's disease), immunodeficiency diseases (eg. HIV), arthritis, atherosclerosis and diabetes;
(c) The monitoring of efficacy for agents used to induce apoptosis in cancers such as: bladder, breast, colon, endometrial, head and neck, leukaemia, lung, melanoma, non-Hodgkins lymphoma, ovarian, prostate and rectal.

### DETAILED DESCRIPTION OF THE INVENTION

In a first aspect, the present invention provides an imaging agent which comprises a synthetic caspase-3 inhibitor labelled with an imaging moiety, wherein the caspase-3 inhibitor has a Kᵢ for caspase-3 of less than 2000 nM, and wherein following administration of said labelled caspase-3 inhibitor to the mammalian body in vivo, the imaging moiety can either be detected externally in a non-invasive manner or by use of detectors designed for use in vivo, such as intravascular radiation or optical detectors (eg. endoscopes), or radiation detectors designed for intra-operative use.

At least fourteen different caspases have been identified in humans to date, which are designated caspase-1, caspase-2 etc. Caspases have been categorised into three main functional categories:

Group I caspases (eg. caspase-1, -4, -5 and -13) which are predominantly involved in the inflammatory response pathway;

Group II caspases (eg. caspase-3, -6, and -7), which are the effector or “executioner” caspases;

Group III caspases (eg. caspase-8, -9 and -2) which are the initiator caspases.

The present invention relates to inhibitors of caspase-3, which is also known as CPP32, and is a 29 kDa cysteine protease.

Suitable imaging agents of the present invention exhibit good cell membrane permeability, and are hence able to target caspase-3, which is an intracellular enzyme. To facilitate cell membrane transport, the imaging agents of the present invention may optionally comprise a “leader peptide” as defined below. Preferred imaging agents do not undergo facile metabolism in vivo, and hence most preferably exhibit a half-life in vivo of 60 to 240 mins in humans. The imaging agent is preferably excreted via the kidney (ie. exhibits urinary excretion). The imaging agent preferably exhibits a signal-to-background ratio at apoptotic foci of at least 1.5, most preferably at least 5, with at least 10 being especially preferred. When the imaging moiety is radioactive, clearance of one half of the peak level of imaging agent which is either non-specifically bound or free in vivo, preferably occurs over a time period less than or equal to the radioactive decay half-life of the radioisotope.

The molecular weight of the imaging agent is suitably up to 5000 Daltons. Preferably, the molecular weight is in the range 150 to 3000 Daltons, most preferably 200 to 1500 Daltons, with 300 to 800 Daltons being especially preferred.

Suitable synthetic caspase-3 inhibitors of the present invention exhibit a K, for caspase-3 of less than 200 nM. Caspase-3 can be expressed in almost all tissues at high levels relative to other caspases, and exhibits high catalytic activity compared to other Group II caspases. Caspase-3 is, however, only expressed in active form during apoptosis. This forms the basis for the labelled inhibitors of the present invention being viable imaging agents with good signal-to-noise. The inhibition constant Kᵢ is the dissociation constant for the enzyme-inhibitor combination [Lehninger, A. L., Nelson, D. L. and Cox, M. M. (1993) Principles of Biochemistry (2nd edn.) Worth, New York Stryer, L. (1995) Biochemistry (4th edn.) Freeman, New York]. Preferably, the inhibitor has a Kᵢ for caspase-3 of less than 500 nM, most preferably less than 100 nM. The synthetic caspase-3 inhibitors of the present invention are also preferably selective for caspase-3 over other caspases. Such selective inhibitors suitably exhibit a greater potency for caspase-3 over caspase-1, defined by Kᵢ, of a factor of at least 50, preferably at least 100, most preferably at least 500.

Preferred synthetic caspase-3 inhibitors of the present invention are irreversible, ie. bind covalently to the enzyme. Since caspase-3 is an intracellular enzyme, preferred caspase-3 inhibitors exhibit good cell membrane permeability, ie. are transported efficiently across mammalian cell membranes in vivo. In this regard, non-peptidic inhibitors are preferred.

The term “labelled with” means that either the caspase-3 inhibitor itself comprises the imaging moiety, or the imaging moiety is attached as an additional species, optionally via a linker group, as described for Formula I below. When the caspase-3 inhibitor itself comprises the imaging moiety, this means that the ‘imaging moiety’ forms part of the chemical structure of the inhibitor, and is a radioactive or non-radioactive isotope present at a level significantly above the natural abundance level of said isotope. Such elevated or enriched levels of isotope are suitably at least 5 times, preferably at least 10 times, most preferably at least 20 times; and ideally either at least 50 times the natural abundance level of the isotope in question, or present at a level where the level of enrichment of the isotope in question is 90 to 100%. Examples of caspase-3 inhibitors comprising the ‘imaging moiety’ are described below, but include CH₃ groups with elevated levels of ¹³C or ¹¹C and fluoroalkyl groups with elevated levels of ¹⁸F, such that the imaging moiety is the isotopically labelled ¹³C, ¹¹C or ¹⁸F within the chemical structure of the caspase-3 inhibitor. The radioisotopes ³H and ¹⁴C are not suitable imaging moieties.

The “imaging moiety” may be detected either external to the mammalian body or via use of detectors designed for use in vivo, such as intravascular radiation or optical detectors such as endoscopes, or radiation detectors designed for intra-operative use. Preferred imaging moieties are those which can be detected externally in a non-invasive manner following administration in vivo. The “imaging moiety” is preferably chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) an optical dye suitable for in vivo imaging.

Most preferred imaging moieties are radioactive, especially radioactive metal ions, gamma-emitting radioactive halogens and positron-emitting radioactive non-metals, particularly those suitable for imaging using SPECT or PET.

When the imaging moiety is a radioactive metal ion, ie. a radiometal. The term “radiometal” includes radioactive transition elements plus lanthanides and actinides, and metallic main group elements. The semi-metals arsenic, selenium and tellurium are excluded from the scope. Suitable radiometals can be either positron emitters such as ⁶⁴Cu, ⁴⁸V, ⁵²Fe, ⁵⁵Co, ^{94m}Tc or ⁶⁸Ga; or γ-emitters such as ^{99m}Tc, ¹¹¹In, ^{113m}In, ⁶⁷Cu or ⁶⁷Ga. Preferred radiometals are ^{99m}Tc, ⁶⁴Cu, ⁶⁴Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(II), Fe(III), Co(II), Er(II), Ni(II), Eu(III) or Dy(I). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I.

When the imaging moiety is a positron-emitting radioactive non-metal, suitable such positron emitters include: ¹¹C, ¹³N, ¹⁷F, ¹⁸F, ⁷⁵Br, ⁷⁶Br or ¹²⁴I. Preferred positron-emitting radioactive non-metals are ¹¹C, ¹³N, ¹²⁴I and ¹⁸F, especially ¹¹C and ¹⁸F, most especially ¹⁸F.

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised. At least one carbon atom of a carbon-containing substituent of the caspase-3 inhibitor of the present invention is suitably enriched with ¹³C, which is subsequently hyperpolarised.

When the imaging moiety is a reporter suitable for in vivo optical imaging, the reporter is any moiety capable of detection either directly or indirectly in an optical imaging procedure. The reporter might be a light scatterer (eg. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, eg. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared region, between 400 nm and 3 μm, particularly between 600 and 1300 nm.

Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

The imaging agents of the present invention are preferably of Formula I: 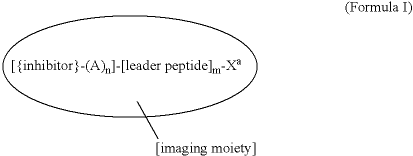
where:
{inhibitor} is the caspase-3 inhibitor of the present invention;
[leader peptide] is a 4 to 20-mer peptide cell membrane transporter peptide, which is conjugated by either its' amine or carboxyl terminus;
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid a sugar or a monodisperse polyethyleneglycol (PEG) building block;
   R is independently chosen from H, C₁₋₄ alkyl, C₂₋₄ alkenyl, C₂₋₄ alkynyl, C₁₋₄ alkoxyalkyl or C₁₋₄ hydroxyalkyl;
   n is an integer of value 0 to 10,
   m is 0 or 1;
   and X^{a} is H, OH, Hal, NH₂, C₁₋₄ alkyl, C₁₋₄ alkoxy, C₁₋₄ alkoxyalkyl, C₁₋₄ hydroxyalkyl or X^{a} is the imaging moiety.

As shown in Formula I, the compounds of the present invention are “labelled with” an imaging moiety. As defined above, this means that one or more of the {inhibitor}, linker group -(A)ₙ or leader peptide either comprises or has conjugated thereto at least one “imaging moiety”. Preferably the caspase-3 inhibitor or the linker group is attached to or comprises the imaging moiety.

The “leader peptide” of the present invention is a 4- to 20-mer peptide which facilitates cell membrane transport. This is important since caspase-3 is an intracellular enzyme, and hence the imaging agents must be capable of crossing cell membranes. The “leader peptide” does not, however, provide biological targeting in vivo. Suitable leader peptides are known in the art, and include: Tat peptides, tachylplesin derivatives and protegrin derivatives. Specific “leader peptide” sequences and references thereto are given below:

TABLE 1

Leader peptides.

| | | |
|---|---|---|
| Leader Peptide | Description | Ref |

| | | | |
|---|---|---|---|
| 1 | CNSRLHLR and | Vascular | Pasqualini R Q |
| | CENWWGDV | targeting | J.Nucl. Med., |
| | | with phage | 43(2):159-62 |
| | | peptide | (1999). |
| | | libraries | |
| 2 | KWSFRVSYRGISYRRSR | Tachylple- | WO 99/07728; |
| | | sin | WO 00/32236; |
| | | derivative | Nakamura et al |
| | | | J Biol Chem. |
| | | | 15; 263(32): |
| | | | 16709-13 |
| | | | (1988).; |
| | | | Tamura H. et |
| | | | al Chem. |
| | | | Pharm. Bull. |
| | | | Tokyo 41, 978- |
| | | | 980 (1993). |
| 3 | AWSFRVSYRGISYRRSR | Tachylple- | WO 99/07728 |
| | | sin | |
| | | derivative | |
| 4 | RKKRRQRRR | TAT | Mie M et al |
| | | | Biochem Bio- |
| | | | phys Res Com- |
| | | | mun. 24; |
| | | | 310(3):730-4 |
| | | | (2003); |
| | | | Potocky TB et |
| | | | al Biol Chem. |
| | | | 2003 Sep 29 |
| | | | [Epub ahead of |
| | | | print] |
| 5 | RRLSYSRRRF | Protegrin | WO 99/07728. |
| | | derivative | |
| 6 | RGGRLSYSRRRFSVSVGR | Protegrin | WO 00/32236; |
| | | | Kokryakov et |
| | | | at FEBS Lett.; |
| | | | 327(2):231-6 |
| | | | (1993). |
| 7 | RGGRLSYSRRRFSTSTGR | Tropic | WO 99/07728; |
| | | protegrin | WO 00/32236. |
| | | (SynB1) | |
| 8 | PRPRPLPFPRPGPPGPRPIPR | Ip (Bac7) | |
| 9 | RQIKIWFQNRRMKWKK | -Penetratin | |
| 10 | RGGGLSYSRRRFSTSTGR | tropic | |
| | | protegrin | |
| 11 | ILPWKWPWWPWRR | Ip | |
| | | (Indolicin) | |
| 12 | FKCRRWQWRMKKLGA | Ip (Lferrin | |
| | | B) | |
| 13 | RLSRIVVIRVSR | Ip | |
| | | (Dodeca- | |
| | | peptide) | |

Preferred “leader peptides” are Tat peptides, tachylplesin derivatives and protegrin derivatives. Most preferred are tachylplesin derivatives and protegrin derivatives.

By the term “amino acid” is meant an L- or D-amino acid, amino acid analogue (eg. napthylalanine) or amino acid mimetic which may be naturally occurring or of purely synthetic origin, and may be optically pure, i.e. a single enantiomer and hence chiral, or a mixture of enantiomers. Preferably the amino acids of the present invention are optically pure.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, eg. a glucosamine derivative of an amino acid can be conjugated to other amino acids via peptide bonds. The glucosamine derivative of asparagine (commercially available from Novabiochem) is one example of this:

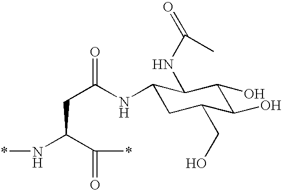

In Formula I, X^{a} is preferably the imaging moiety. This has the advantage that the linker group -(A)ₙ- of Formula I distances the imaging moiety from the active site of the metalloproteinase inhibitor. This is particularly important when the imaging moiety is relatively bulky (eg. a metal complex or a radioiodine atom), so that binding of the inhibitor to the caspase enzyme is not impaired. This can be achieved by a combination of flexibility (eg. simple alkyl chains), so that the bulky group has the freedom to position itself away from the active site and/or rigidity such as a cycloalkyl or aryl spacer which orientates the metal complex away from the active site

The nature of the linker group can also be used to modify the biodistribution of the imaging agent. Thus, eg. the introduction of ether groups in the linker will help to minimise plasma protein binding. When -(A)ₙ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. via the kidneys as opposed to via the liver.

When -(A)ₙ- comprises a peptide chain of 1 to 10 amino acid residues, the amino acid residues are preferably chosen from glycine, lysine, aspartic acid, glutamic acid or serine. When -(A)ₙ- comprises a PEG moiety, it preferably comprises units derived from oligomerisation of the monodisperse PEG-like structures of Formulae IIA or IIB:

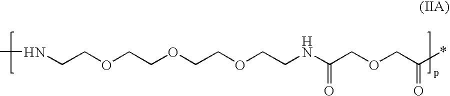

17-amino-5-oxo-6-aza-3,9,12,15-tetraoxaheptadecanoic acid of Formula IIA wherein p is an integer from 1 to 10 and where the C-terminal unit (*) is connected to the imaging moiety. Alternatively, a PEG-like structure based on a propionic acid derivative of Formula IIB can be used: 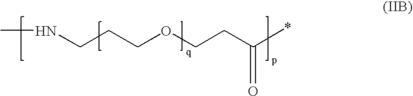
where p is as defined for Formula IIA and q is an integer from 3 to 15.

In Formula IIB, p is preferably 1 or 2, and q is preferably 5 to 12.

When the linker group does not comprise PEG or a peptide chain, preferred (A)ₙ- groups have a backbone chain of linked atoms which make up the -(A)ₙ- moiety of 2 to 10 atoms, most preferably 2 to 5 atoms, with 2 or 3 atoms being especially preferred. A minimum linker group backbone chain of 2 atoms confers the advantage that the imaging moiety is well-separated from the caspase-3 inhibitor so that any interaction is minimised.

Non-peptide linker groups such as alkylene groups or arylene groups have the advantage that there are no significant hydrogen bonding interactions with the conjugated caspase-3 inhibitor, so that the linker does not wrap round onto the inhibitor. Preferred alkylene spacer groups are —(CH₂)_{q}— where q is 2 to 5. Preferred arylene spacers are of formula: 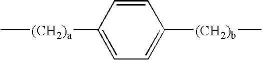
where: a and b are independently 0, 1 or 2.

The linker group -(A)ₙ- preferably comprises a diglycolic acid moiety, a maleimide moiety, a glutaric acid, succinic acid, a polyethyleneglycol based unit or a PEG-like unit of Formula IIA.

When the imaging moiety comprises a metal ion, the metal ion is present as a metal complex. Such caspase-3 inhibitor conjugates with metal ions are therefore suitably of Formula Ia: 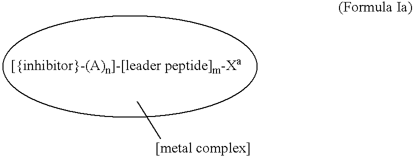
where: A, n, m and X^{a} are as defined for Formula I above.

By the term “metal complex” is meant a coordination complex of the metal ion with one or more ligands. It is strongly preferred that the metal complex is “resistant to transchelation”, ie. does not readily undergo ligand exchange with other potentially competing ligands for the metal coordination sites. Potentially competing ligands include the caspase-3 inhibitor itself plus other excipients in the preparation in vitro (eg. radioprotectants or antimicrobial preservatives used in the preparation), or endogenous compounds in vivo (eg. glutathione, transferrin or plasma proteins). The metal complex is preferably attached at the linker group -(A)ₙ- or at one of the amino acid residues of the leader peptide. The metal complex is most preferably attached at one of the A residues furthest distant from the inhibitor, such that a leader peptide can also be present by either attachment at the terminal A residue of the linker group, or by branching from a non-terminal A residue.

The metal complexes of Formula Ia are derived from conjugates of ligands of Formula Ib: 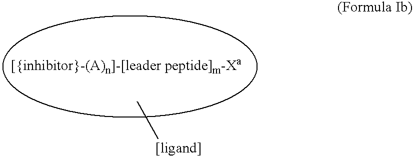
where: A, n, m and X^{a} are as defined for Formula I above.

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:
(i) diaminedioximes of formula: 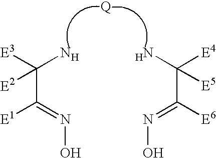
where E¹-E⁶ are each independently an R′ group;
each R′ is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R′ groups together with the atoms to which they are attached form a carbocyclic, heterocyclic, saturated or unsaturated ring, and wherein one or more of the R′ groups is conjugated to the caspase-3 inhibitor;
and Q is a bridging group of formula -(J)_{f}-;
where f is 3, 4 or 5 and each J is independently —O—, —NR′— or —C(R′)₂— provided that -(J)_{f}- contains a maximum of one J group which is —O— or —NR′—.

Preferred Q groups are as follows:

Q=—(CH₂)(CHR′)(CH₂)— ie. propyleneamine oxime or PnAO derivatives;

Q=—(CH₂)₂(CHR′)(CH₂)₂— ie. pentyleneamine oxime or PentAO derivatives;

Q=—(CH₂)₂NR′(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The caspase-3 inhibitor is preferably conjugated at either the E¹ or E⁶ R′ group, or an R′ group of the Q moiety. Most preferably, the caspase-3 inhibitor is conjugated to an R′ group of the Q moiety. When the caspase-3 inhibitor is conjugated to an R′ group of the Q moiety, the R′ group is preferably at the bridgehead position. In that case, Q is preferably —(CH₂)(CHR′)(CH₂)—, —(CH₂)₂(CHR′)(CH₂)₂— or —(CH₂)₂NR′(CH₂)₂—, most preferably —(CH₂)₂(CHR′)(CH₂)₂—.

An especially preferred bifunctional diaminedioxime chelator is Chelator 1: 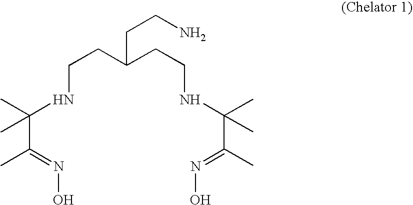
such that the caspase-3 inhibitor is conjugated via the bridgehead —CH₂CH₂NH₂ group.
(ii) N₃S ligands having a thioltriamide donor set such as MAG3 (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam.
(v) N₂O₂ ligands having a diaminediphenol donor set.

The above described ligands are particularly suitable for complexing technetium eg. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷Cu), vanadium (eg. ⁴⁸V), iron (eg. ⁵²Fe), or cobalt (eg. ⁵⁵Co). Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above.

When the imaging moiety is a radioactive halogen, such as iodine, the caspase-3 inhibitor is suitably chosen to include: a non-radioactive precursor halogen atom such as an aryl iodide or bromide (to permit radioiodine exchange); an activated precursor aryl ring (e.g. a phenol group); an organometallic precursor compound (eg. trialkyltin or trialkylsilyl); or an organic precursor such as triazenes or a good leaving group for nucleophilic substitution such as an iodonium salt. Methods of introducing radioactive halogens (including ¹²³I and ¹⁸F are described by Bolton [J. Lab. Comp. Radiopharm., 45, 485-528 (2002)]. Examples of suitable precursor aryl groups to which radioactive halogens, especially iodine can be attached are given below:

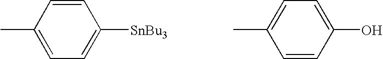

Both contain substituents which permit facile radioiodine substitution onto the aromatic ring. Alternative substituents containing radioactive iodine can be synthesised by direct iodination via radiohalogen exchange, e.g.

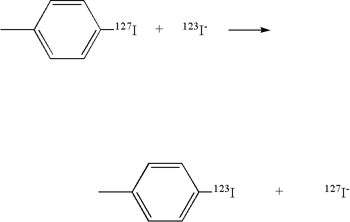

When the imaging moiety is a radioactive isotope of iodine the radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

When the imaging moiety comprises a radioactive isotope of fluorine (eg. ¹⁸F), the radiohalogenation may be carried out via direct labelling using the reaction of ¹⁸F-fluoride with a suitable precursor having a good leaving group, such as an alkyl bromide, alkyl mesylate or alkyl tosylate. ¹⁸F can also be introduced by N-alkylation of amine precursors with alkylating agents such as ¹⁸F(CH₂)₃OMs (where Ms is mesylate) to give N—(CH₂)₃¹⁸F, or O-alkylation of hydroxyl groups with ¹⁸F(CH₂)₃OMs or ¹⁸F(CH₂)₃Br. ¹⁸F can also be introduced by alkylation of N-haloacetyl groups with a ¹⁸F(CH₂)₃OH reactant, to give —NH(CO)CH₂O(CH₂)₃¹⁸F derivatives. For aryl systems, ¹⁸F-fluoride nucleophilic displacement from an aryl diazonium salt, aryl nitro compound or an aryl quaternary ammonium salt are suitable routes to aryl-¹⁸F derivatives.

Primary amine-containing caspase-3 inhibitors can also be labelled with ¹⁸F by reductive amination using ¹⁸F—C₆H₄—CHO as taught by Kahn et al [J. Lab. Comp. Radiopharm. 45, 1045-1053 (2002)] and Borch et al [J. Am. Chem. Soc. 93, 2897 (1971)]. This approach can also usefully be applied to aryl primary amines, such as compounds comprising phenyl-NH₂ or phenyl-CH₂NH₂ groups. For peptide-based inhibitors which do not also contain a haloalkylketone functional group, this approach can be applied to aminoxy derivatives of peptides as taught by Poethko et al [J. Nuc. Med., 45, 892-902 (2004)].

Amine-containing caspase-3 inhibitors can also be labelled with ¹⁸F by reaction with ¹⁸F-labelled active esters such as: 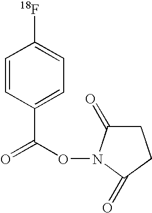
to give amide bond linked products. The N-hydroxysuccinimide ester shown and its use to label peptides is taught by Vaidyanathan et al [Nucl. Med. Biol., 19(3), 275-281 (1992)] and Johnstrom et al [Clin. Sci., 103 (Suppl. 48), 45-85 (2002)]. Further details of synthetic routes to ¹⁸F-labelled derivatives are described by Bolton, J. Lab. Comp. Radiopharm., 4, 485-528 (2002).

For maximum sensitivity in vivo it is most preferred that the imaging moiety comprises a radioactive element. The imaging moiety preferably comprises a positron-emitting or a gamma-emitting radioisotope.

The synthetic caspase-3 inhibitors of the present invention are preferably selected from the following:
(i) a tetrapeptide derivative of Formula III:
   Z¹-Asp-Xaa1-Xaa2-Asp-X¹ (III)
where Z¹ is a metabolism inhibiting group attached to the N-terminus of the tetrapeptide;
   Xaa1 and Xaa2 are independently any amino acid;
   Asp is the conventional three letter abbreviation for aspartic acid;
   X¹ is an —R¹ or —CH₂OR² group attached to the carboxy terminus of the tetrapeptide;
   where R¹ is H, —CH₂F, —CH₂Cl, C₁₋₅ alkyl, C₁₋₅ alkoxy or —(CH₂)_{q}Ar¹, where q is an integer of value 1 to 6 and Ar¹ is C₆₋₁₂ aryl, C₅₋₁₂ alkyl-aryl, C₅₋₁₂ fluoro-substituted aryl, or C₃₋₁₂ heteroaryl;
   R² is C₁₋₅ alkyl, C₁₋₁₀ acyl or Ar¹;
(ii) a quinazoline or anilinoquinazoline;
(iii) a 2-oxindole sulphonamide;
(iv) an oxoazepinoindoline;
(v) a compound of Formula IV: 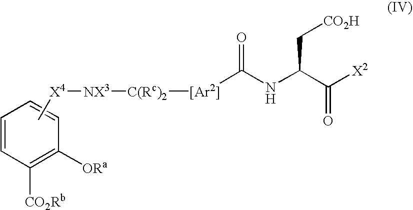
   where X² is H, C₁₋₅ alkyl or —(CH₂)ᵣ—(S)ₛ—(CH₂)ₜAr³, where r and t are integers of value 0 to 6, s is 0 or 1 and Ar³ is C₆₋₁₂ aryl, C₅₋₁₂ alkyl-substituted aryl, C₅₋₁₂ halo-substituted aryl, or C₃₋₁₂ heteroaryl;
   Ar² is C₆₋₁₂ aryl or C₃₋₁₂ heteroaryl;
   X³ is an R^{b} group;
   X⁴ is —SO₂— or —CR₂—
   R^{a} is H, C₁₋₅ alkyl or p^{GP} where p^{GP} is a protecting group;
   R^{b} is an R^{a} group or C₁₋₅ acyl;
   each R^{c} is independently H or C₁₋₅ alkyl;
(vi) a compound of Formula V: 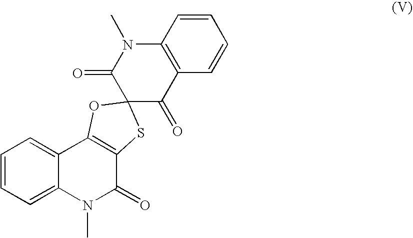
(vii) a pyrazinone;
(viii) a dipeptide of Formula VI:
   Z¹-Val-Asp-CH₂—S—R′ (VI)
   where the —CH₂—S—R¹ group is attached to the carboxy terminus of the dipeptide, and Z¹ and R¹ are as defined for Formula (III).
(ix) A salicylic acid sulphonamide of Formula XI: 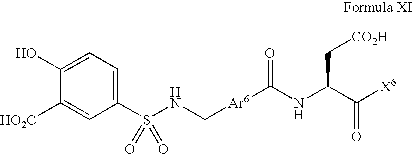
   Where Ar⁶ is a 5 or 6-membered C₄₋₆ aryl or heteroaryl ring, and X6 is H or —CH₂SR², where R2 is as defined above.

The term “amino acid” is as defined above.

Peptide aldehyde (X¹=R¹=H), ketone [X¹=R¹=C₁₋₅ alkyl or —(CH₂)_{q}Ar¹] or phenoxymethylketone (X¹=—CH₂OR² and R²=Ar¹=phenyl) inhibitors of Formula III are reversible caspase inhibitors, whereas chloromethyl and fluoromethyl derivatives (X¹=R¹=—CH₂F or —CH₂Cl), plus acyloxymethylketones (X¹=—CH₂OR² and R²=C₁₋₁₀ acyl) are irreversible inhibitors. The halomethylketone peptides are believed to bind to the cysteine thiol of caspase-3, forming a thiomethyl ketone and thus irreversibly inactivating the enzyme. As indicated above, such irreversible inhibitors are preferred. Hence, X¹ of Formula III is preferably —CH₂F or —CH₂OR² with R²=C₁₋₁₀ acyl. When R² is C₁₋₁₀ acyl, a preferred such acyl group is 2,6-disubstituted benzoyl such as (2,6-dimethylphenyl)(C═O)— or [2,6-bis(trifluoromethyl)phenyl](C═O).

By the term “metabolism inhibiting group” (Z¹) is meant a biocompatible group which inhibits or suppresses in vivo metabolism of the peptide or amino acid at the amino terminus. Such groups are well known to those skilled in the art and are suitably chosen from, for the peptide amine terminus: acetyl, Boc (where Boc is tert-butyloxycarbonyl), Fmoc (where Fmoc is fluorenylmethoxycarbonyl), benzyloxycarbonyl, trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). A preferred metabolism inhibiting group for the peptide N-terminus is acetyl.

In Formula III, Xaa1 and Xaa2 are most preferably any L-amino acid. Xaa1-Xaa2 is preferably Glu-Val or Gln-Met, so that preferred compounds of Formula III are: Z¹-Asp-Glu-Val-Asp-X¹ or Z¹-Asp-Gln-Met-Asp-X¹ (ie. Z¹-DEVD-X¹ or Z¹-DQMD-X¹).

In Formula III, the carboxy group of the aspartyl and glutamyl side chain is preferably present as the free carboxylate so that the caspase-3 inhibitor is potent. However, the carboxy group can also be present as an ester, e.g. methyl ester to improve cell permeability. The ester is subsequently deprotected by esterases present in the non-necrotic cells. For Formula III, the imaging moiety is preferably attached at the Z¹ or X¹ positions. When the imaging moiety comprises a metal, inhibition of metabolism of the peptide amine or carboxyl terminus of the peptide of Formula III is preferably achieved by attachment of either or both termini to a metal complex of the metal.

By the term “protecting group” (P^{GP}) is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: benzyl, acetyl, benzoyl, trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Some caspase-3 inhibitors of Formula III are commercially available, eg. Ac-DEVD-CHO, Ac-AAVALLPAVLLALLAP-DEVD-CHO, Z-DEVD-FMK, and Ac-DEVD-CMK, which can be purchased from Calbiochem through VWR INTERNATIONAL LTD. Hunter Boulevard, Magna Park, Lutterworth LE17 4XN UNITED KINGDOM. Others can be prepared as described by Thornberry et al [J. Biol. Chem., 272 (29), 17907-17911 (1997); ibid, 273 (49), 32608-32613 (1998)]. Peptide-containing caspase-3 inhibitors and leader peptides of the present invention may also be obtained by conventional solid phase synthesis, as described in P. Lloyd-Williams, F. Albericio and E. Girald; Chemical Approaches to the Synthesis of Peptides and Proteins, CRC Press, 1997.

Quinazoline or anilinoquinazoline caspase-3 inhibitors are described by Scott et al [J. Pharmacol. Exper. Ther., 304(1), 433-440 (2003)]. Preferred such compounds have the general Formula VII: 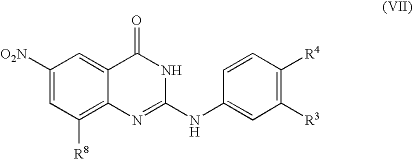
where:
R³ is H or Cl;
R⁴ is Cl or F;
R⁸ is —CONH—X⁵ or —CH═CH—Ar⁴, where X⁵ is C₁₋₆ alkyl, C₂₋₆ alkenyl or —(CH₂)ₛAr⁴; where s is 0 or 1, Ar⁴ is —C₆H₅X⁶ and X⁶ is Hal, CF₃ or —SO₂NR⁶R⁷.
   R⁶ and R⁷ are independently C₁₋₃ alkyl, or may be combined to form a C₅₋₇ cycloalkyl ring.
      R⁸ is preferably —CONH—X⁵ with X⁵=—(CH₂)ₛAr⁴. X⁶ is preferably F, CF₃ or —SO₂NC₆H₁₀.

Preferred 2-oxindole sulphonamide derivatives of the present invention are of Formula VIII: 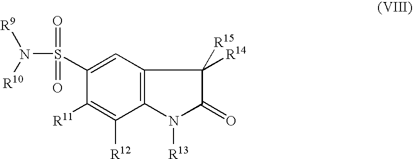
where:
R⁹ is H or C₁₋₄ alkyl;
R¹⁰ is C₁₋₁₀ alkyl, arylC₁₋₄ alkyl, heteroaryl C₁₋₄ alkyl C₃₋₇ cycloalkyl, or R⁹ and R¹⁰ together with the nitrogen atom to which they are attached form a 3 to 10-membered ring which optionally contains a further heteroatom selected from O, N or S;
R¹¹ and R¹² are independently H, C₁₋₆ alkyl, NO₂ or Hal;
R¹³ is H, C₁₋₆ alkyl, C₆₋₁₂ arylalkyl or C₃₋₁₂ heteroarylalkyl;
R¹⁴ and R¹⁵ are Cl or together with the carbon atom to which they are attached form a C═O carbonyl group.

In Formula VIII, R¹⁴ and R¹⁵ are preferably together equal to C═O, ie. an isatin derivative. R¹³ is preferably H or CH₃. R⁹ and R¹⁰ are preferably C₄₋₆ cycloalkyl, most preferably C₅ cycloalkyl. When R⁹ and R¹⁰ are C₄₋₆ cycloalkyl, the cycloalkyl ring is preferably substituted with an X⁷ group, where X⁷ is —CH₂OR¹⁶ or —H₂NHR¹⁶ and R¹⁶ is C₁₋₃ alkyl or C₄₋₇ aryl. The 2-oxindole sulphonamide derivatives of Formula VIII can be prepared as described by Lee et al [J. Biol. Chem., 275, 16007-16014 (2000)].

The imaging moiety is preferably attached to the R⁹, R¹⁰ R¹¹, R¹² or R¹³ substituents of the inhibitors of Formula VIII, most preferably the R⁹, R¹⁰ or R¹³ substituents. For ¹⁸F labelling, R¹³ is chosen to be either —CH₂ONH₂ for the 4-¹⁸F-benzaldehyde imine route (described above), or is —CH₂OH for labelling with ¹⁸F—(CH₂)₃Br or ¹⁸F—(CH₂)₃OTs type O-alkylating agents. Alternatively, R¹³ is chosen to be H, so that direct N-alkylation leads to the desired ¹⁸F derivatives.

A preferred oxoazepinoindoline of the present invention is IDN5370, which is shown in Formula IX:

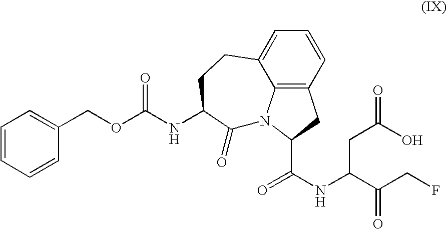

A most preferred oxoazepinoindoline is Indun A:

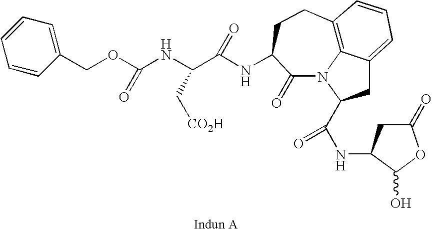

Oxoazepinoindolines of the present invention are described by Deckwerth et al [Drug Devel. Res., 2, 579-586 (2001)], and in WO 98/11109.

Pyrazinones of the present invention are suitably of Formula X: 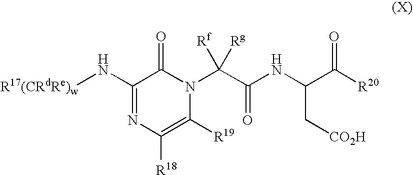
where:
R¹⁷ is OH, NH₂, NHRⁱ, N(Rⁱ)₂, Rⁱ, C₁₋₆ alkoxy, Ar⁵, Het¹, X⁸(CO)—, X⁸SO— or X⁸SO₂—, where each R¹ is independently C₁₋₆ alkyl, which may optionally be substituted by 1 to 3 substituents chosen from OH, Hal, CO₂H, CF₃, NH₂, NHCH₃, N(CH₃)₂, Ar⁵ and C₁₋₄ acyl,
   Ar⁵ is a C₆₋₁₄ aromatic ring which may optionally be substituted by 1 to 3 OH, Hal, CO₂H, CF₃, NH₂, NHCH₃, N(CH₃)₂, C₁₋₆ alkyl, C₁₋₆ alkoxy, Het¹ or C₁₋₄ acyl substituents, and
   X⁸ is Rⁱ, Ar⁵ or Het¹;
   Het¹ is a 5 to 15-membered heterocyclic or heteroaryl ring containing 1 to 4 heteroatoms chosen from O, S and N, which may be optionally substituted with one or two oxo groups, and 1 to 3 groups chosen from C₁₋₄ alkyl, C₁₋₄ alkoxy, C₁₋₄ acyl and CF₃;
R¹⁸ is H, C₁₋₂₀ alkyl, Ar⁵ or Het¹;
R¹⁹ is H, Hal or C₁— alkyl;
R²⁰ is H, C₁₋₆ alkyl, Ar⁵, Het¹, —(CH₂)_{z}SRⁱ, —(CH₂)_{z}ORⁱ, —(CH₂)_{z}OC(O)R^{j} or —(CH₂)_{z}NR²¹R²² where z is 1, 2 or 3;
   R^{j} is C₁₋₈ alkyl, Ar⁵ or Het¹; and
   R²¹ and R²² are independently H, Rⁱ, Ar⁵ or Het¹, or R²¹ and R²² taken together with the nitrogen atom to which they are attached form a 3 to 10-membered ring system containing 1 to 4 heteroatoms chosen from O, S, and N which may be optionally substituted with one or two oxo groups, and 1 to 3 groups chosen from C₁₋₄ alkyl, Het¹, C₁₋₄ carboxy, C₁₋₄ acyl and C₁₋₆ carboxamide;
R^{d} and R^{e} are independently H, C₁₋₆ alkyl or Ar⁵ or may be combined with the carbon atom to which they are attached to form a 3 to 7-membered non-aromatic alicyclic or heterocyclic ring optionally containing one heteroatom chosen from O, S and NR²³, where R²³ is H, C₁₋₄ alkyl or C₁₋₄ acyl;
R^{f} and R^{g} are independently H, Ar⁵, C₁₋₆ alkyl, C₁₋₆ alkoxyalkyl, or C₅₋₇ cycloalkyl;
w is an integer of value 0 to 6.

A preferred pyrazinone which is selective for caspase-3 is L-826,791 or M-826 [Hotchkiss et al, Nature Immunol., 1(6), 496-501 (2000)]:

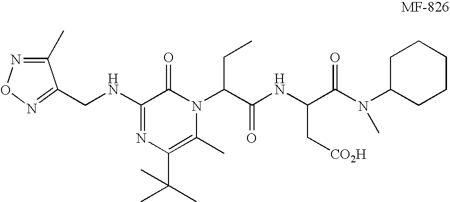

The synthesis of pyrazinone caspase-3 inhibitors of the invention is described in U.S. Pat. No. 6,444,811, and is shown in Scheme 1 (overleaf). The starting material is dimethylglyoxime which is commercially available.

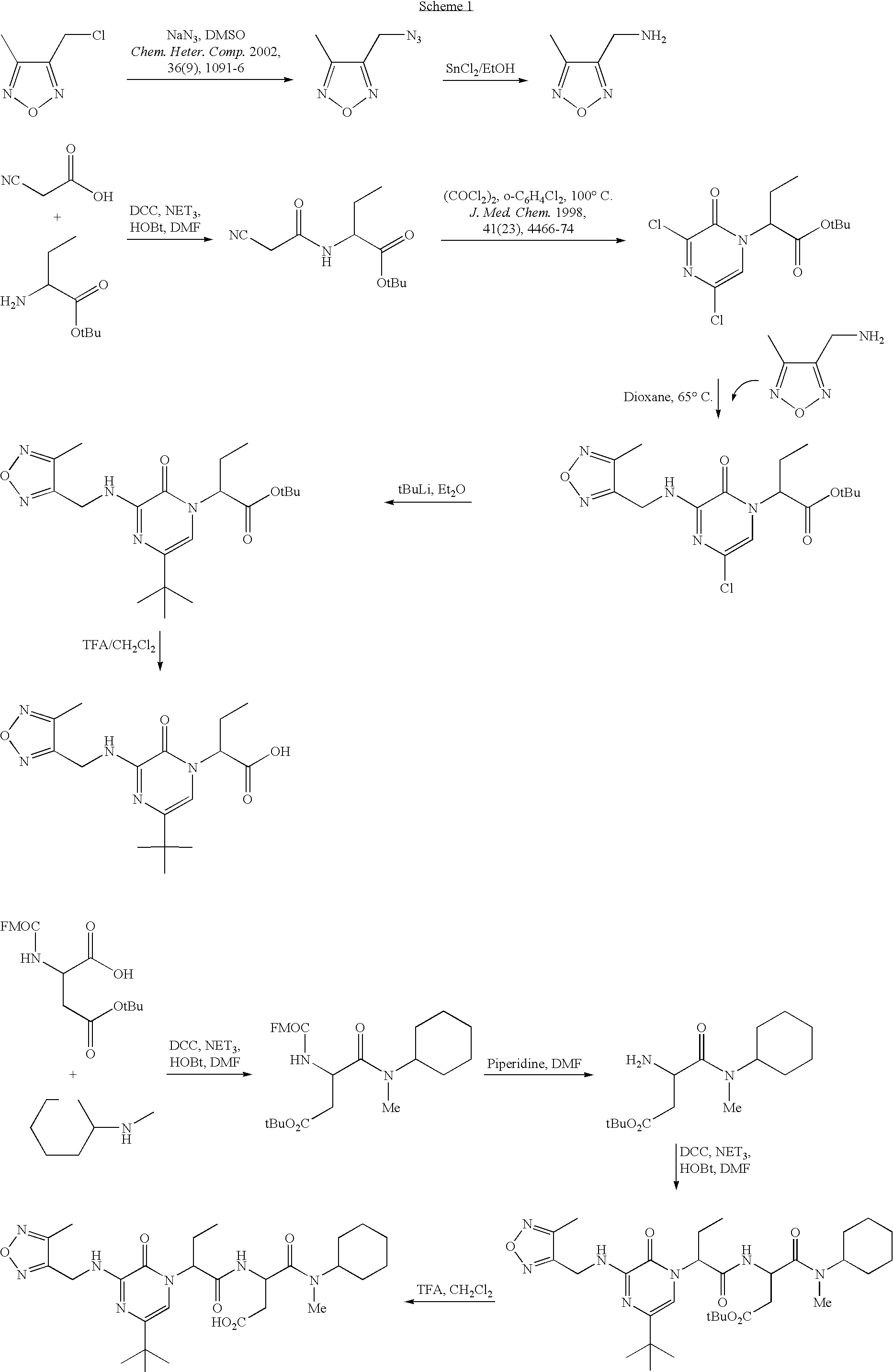

Compounds of Formula V can be prepared as described in WO 03/024955. Compounds of Formula VI can be prepared as shown in Scheme 2:

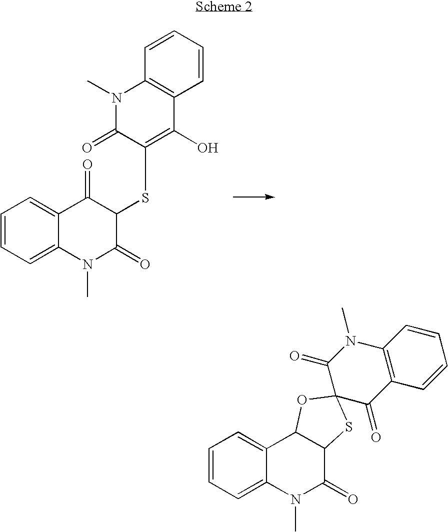

The dipeptide inhibitors of Formula VI are aspartyl ketones and are described by Han et al [Bioorg. Med. Chem. Lett. 2004, X, 805-808)]. These are potent and selective caspase-3 inhibitors. For Formula VI, the imaging moiety is preferably attached at the Z¹ or X¹ positions. Preferred caspase-3 inhibitors of Formula VI are of Formula VIa: 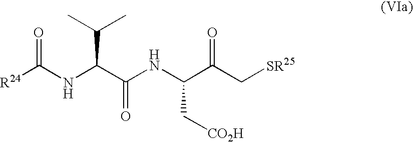
where R²⁴ is C₆₋₁₂ aryl or C₆₋₁₂ heteroaryl;
and R²⁵ is C₁₋₄ alkyl or benzyl, where the phenyl ring of the benzyl group is optionally substituted by 1 or 2 halogen atoms;
R²⁴ is preferably a benzyl group where the phenyl ring of the benzyl group is optionally substituted by 1 or 2 groups chosen from: halogen, C₁₋₃ alkoxy; C₁₋₃ alkoxy substituted with a C₁₋₃ carboxyl or C₂₋₄ carboxyester group; C₁₋₃ acyl; C₂₋₄ alkenyl or C₁₋₃ alkylsulfonyl.
R²⁵ is preferably a benzyl group or a 2-chloro-5-fluoro-benzyl group.

Especially preferred inhibitors of Formula VI contain a substituted 2-chloro-6-fluorobenzyl group at R¹ and a 2,5-disubstituted benzylcarbonyl at Z¹. These are of Formula VIb:

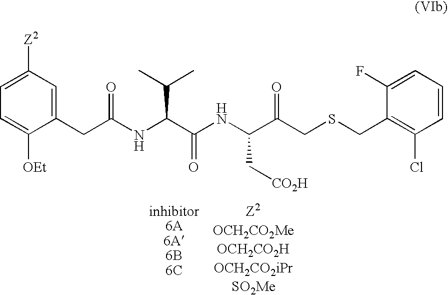

Inhibitors 6A, 6A′, 6B and 6C exhibit IC₅₀ for caspase-3 in the low nanomolar range [Han et al Bioorg. Med. Chem. Lett. 2004, 14, 805-808)]. The ester derivatives 6A and 6B are hydrolysed intracellularly to the more potent acid 6A′.

The synthesis of the compounds of Formula VI, and most preferred potent caspase-3 inhibitors based thereon are described by Han et al [Bioorg. Med. Chem. Lett., 14(3), 805-808 (2004)]. The imaging moiety is preferably attached at either of the phenyl rings of Formula VIb, most preferably at the Z² position. It is envisaged that an ¹⁸F label could be introduced as shown in Scheme 3:

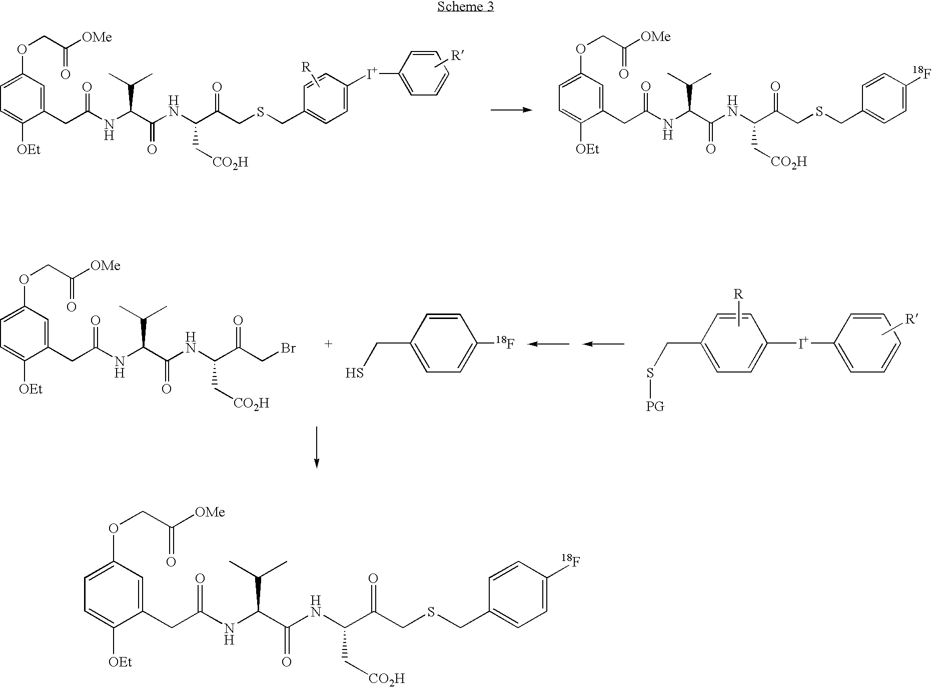

Inhibitors of Formula XI can be prepared by the method of Choong et al. [J. Med. Chem. 45, 5005-5022 (2002)]; Erlanson et al. [Nature Biotech., 21, 308-314 (2003)] or of WO 03/024955. Preferred inhibitors of Formula XI have Ar⁶ chosen from phenyl, thiophene, or pyridine; especially thiophene. In Formula X¹, X⁶ is preferably —CH₂SAr⁷, where Ar⁷ is a halogen-substituted phenyl ring. A preferred inhibitor of Formula XI is of Formula XIa:

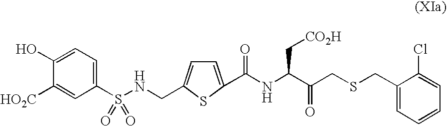

Preferred caspase-3 inhibitors of the present invention are the tetrapeptides of Formula III, dipeptides of Formula VI or 2-oxindole sulphonamides of Formula VIII. Most preferred inhibitors are the tetrapeptides of Formula III, and the dipeptides of Formula VI.

When the imaging agent of the present invention comprises a radioactive or paramagnetic metal ion, the metal ion is suitably present as a metal complex. Such metal complexes are suitably prepared by reaction of the conjugate of Formula Ia with the appropriate metal ion. The ligand-conjugate or chelator-conjugate of the caspase-3 inhibitor of Formula Ia can be prepared via the bifunctional chelate approach. Thus, it is well known to prepare ligands or chelating agents which have attached thereto a functional group (“bifunctional linkers” or “bifunctional chelates” respectively). Functional groups that have been attached include: amine, thiocyanate, maleimide and active esters such as N-hydroxysuccinimide or pentafluorophenol. Chelator 1 of the present invention is an example of an amine-functionalised bifunctional chelate. Such bifunctional chelates can be reacted with suitable functional groups on the caspase-3 inhibitor to form the desired conjugate. Such suitable functional groups on the caspase-3 inhibitor include: carboxyls (for amide bond formation with an amine-functionalised bifunctional chelator); amines (for amide bond formation with an carboxyl- or active ester-functionalised bifunctional chelator);

halogens, mesylates and tosylates (for N-alkylation of an amine-functionalised bifunctional chelator) and

thiols (for reaction with a maleimide-functionalised bifunctional chelator).

The radiometal complexes of the present invention may be prepared by reacting a solution of the radiometal in the appropriate oxidation state with the ligand conjugate of Formula Ia at the appropriate pH. The solution may preferably contain a ligand which complexes weakly to the metal (such as gluconate or citrate) i.e. the radiometal complex is prepared by ligand exchange or transchelation. Such conditions are useful to suppress undesirable side reactions such as hydrolysis of the metal ion. When the radiometal ion is ^{99m}Tc, the usual starting material is sodium pertechnetate from a ⁹⁹Mo generator. Technetium is present in ^{99m}Tc-pertechnetate in the Tc(VII) oxidation state, which is relatively unreactive. The preparation of technetium complexes of lower oxidation state Tc(I) to Tc(V) therefore usually requires the addition of a suitable pharmaceutically acceptable reducing agent such as sodium dithionite, sodium bisulphite, ascorbic acid, formamidine sulphinic acid, stannous ion, Fe(II) or Cu(I), to facilitate complexation. The pharmaceutically acceptable reducing agent is preferably a stannous salt, most preferably stannous chloride, stannous fluoride or stannous tartrate.

When the imaging moiety is a hyperpolarised NMR-active nucleus, such as a hyperpolarised ¹³C atom, the desired hyperpolarised compound can be prepared by polarisation exchange from a hyperpolarised gas (such as ¹²⁹Xe or ³He) to a suitable ¹³C-enriched caspase-3 inhibitor.

In a second aspect, the present invention provides a pharmaceutical composition which comprises the imaging agent as described above, together with a biocompatible carrier, in a form suitable for mammalian administration. The “biocompatible carrier” is a fluid, especially a liquid, in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like).

In a third aspect, the present invention provides a radiopharmaceutical composition which comprises the imaging agent as described above wherein the imaging moiety is radioactive, together with a biocompatible carrier (as defined in the second embodiment above), in a form suitable for mammalian administration. Such radiopharmaceuticals are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, and are therefore preferably a disposable or other syringe suitable for clinical use. The pre-filled syringe may optionally be provided with a syringe shield to protect the operator from radioactive dose. Suitable such radiopharmaceutical syringe shields are known in the art and preferably comprise either lead or tungsten.

When the imaging moiety comprises ^{99m}Tc, a radioactivity content suitable for a diagnostic imaging radiopharmaceutical is in the range 180 to 1500 MBq of ^{99m}Tc, depending on the site to be imaged in vivo, the uptake and the target to background ratio.

The radiopharmaceuticals of the present invention may be prepared from kits, as is described in the fifth and sixth embodiments below. Alternatively, the radiopharmaceuticals may be prepared under aseptic manufacture conditions to give the desired sterile product. The radiopharmaceuticals may also be prepared under non-sterile conditions, followed by terminal sterilisation using e.g. gamma-irradiation, autoclaving, dry heat or chemical treatment (e.g. with ethylene oxide). Preferably, the radiopharmaceuticals of the present invention are prepared from kits.

In a fourth aspect, the present invention provides a conjugate of the synthetic caspase-3 inhibitor of the invention with a ligand. Said conjugates are useful for the preparation of synthetic caspase-3 inhibitors labelled with either a radioactive metal ion or paramagnetic metal ion. Preferably, the ligand conjugate is of Formula Ia, as defined above. The ligand of the conjugate of the fourth aspect of the invention is preferably a chelating agent. Preferably, the chelating agent has a diaminedioxime, N₂S₂ diaminedithiol or N₃S diamidepyridinethiol donor set. Most preferably, the chelating agent is a diaminedioxime.

In a fifth aspect, the present invention provides a non-radioactive kit for the preparation of the radiopharmaceutical composition described above where the imaging moiety comprises a radiometal. The kit comprises a conjugate of a ligand with the caspase-3 inhibitor of Formula (I). When the radiometal is ^{99m}Tc, the kit suitably further comprises a biocompatible reductant. The ligand conjugates, and preferred aspects thereof, are described in the fourth embodiment above.

Such kits are designed to give sterile radiopharmaceutical products suitable for human administration, e.g. via direct injection into the bloodstream. For ^{99m}Tc, the kit is preferably lyophilised and is designed to be reconstituted with sterile ^{99m}Tc-pertechnetate (TcO₄⁻) from a ^{99m}Tc radioisotope generator to give a solution suitable for human administration without further manipulation. Suitable kits comprise a container containing the ligand or chelator conjugate in either free base or acid salt form, together with a “biocompatible reductant” such as sodium dithionite, sodium bisulphite, ascorbic acid, formamidine sulphinic acid, stannous ion, Fe(II) or Cu(I). The biocompatible reductant is preferably a stannous salt such as stannous chloride or stannous tartrate. Alternatively, the kit may optionally contain a metal complex which, upon addition of the radiometal, undergoes transmetallation (i.e. metal exchange) giving the desired product.

Suitable kit containers comprise a sealed container which permits maintenance of sterile integrity and/or radioactive safety, plus optionally an inert headspace gas (eg. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired eg. to change the headspace gas or degas solutions.

The non-radioactive kits may optionally further comprise additional components such as a transchelator, radioprotectant, antimicrobial preservative, pH-adjusting agent or filler. The “transchelator” is a compound which reacts rapidly to form a weak complex with the radiometal, then is displaced by the ligand. For technetium, this minimises the risk of formation of reduced hydrolysed technetium (RHT) due to rapid reduction of pertechnetate competing with technetium complexation. Suitable such transchelators are salts of a weak organic acid, ie. an organic acid having a pKa in the range 3 to 7, with a biocompatible cation. Suitable such weak organic acids are acetic acid, citric acid, tartaric acid, gluconic acid, glucoheptonic acid, benzoic acid, phenols or phosphonic acids. Hence, suitable salts are acetates, citrates, tartrates, gluconates, glucoheptonates, benzoates, phenolates or phosphonates. Preferred such salts are tartrates, gluconates, glucoheptonates, benzoates, or phosphonates, most preferably phosphonates, most especially diphosphonates. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium. A preferred such transchelator is a salt of MDP, ie. methylenediphosphonic acid, with a biocompatible cation.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (ie. 4-aminobenzoic acid), gentisic acid (ie. 2,5-dihydroxybenzoic acid) and salts thereof with a biocompatible cation as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, ie. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

In a sixth aspect, the present invention provides kits for the preparation of radiopharmaceutical preparations where the imaging moiety comprises a non-metallic radioisotope, ie. a gamma-emitting radioactive halogen or a positron-emitting radioactive non-metal. Such kits comprise a “precursor”, preferably in sterile non-pyrogenic form, so that reaction with a sterile source of the radioisotope gives the desired radiopharmaceutical with the minimum number of manipulations. Such considerations are particularly important for radiopharmaceuticals where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

The “precursor” suitably comprises a non-radioactive derivative of the caspase-3 inhibitor material in sterile, apyrogenic form, which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors can conveniently be obtained in good chemical purity. The “precursor” may optionally comprise a protecting group (P^{GP}), as defined above, for certain functional groups of the caspase-3 inhibitor. Suitable precursors are described by Bolton, J. Lab. Comp. Radiopharm., 45, 485-528 (2002).

Preferred precursors of this embodiment comprise a derivative which either undergoes electrophilic or nucleophilic halogenation; undergoes facile alkylation with an alkylating agent chosen from an alkyl or fluoroalkyl halide, tosylate, triflate (ie. trifluoromethanesulphonate) or mesylate; or alkylates thiol moieties to form thioether linkages. Examples of the first category are:
(a) organometallic derivatives such as a trialkylstannane (eg. trimethylstannyl or tributylstannyl), or a trialkylsilane (eg. trimethylsilyl);
(b) a non-radioactive alkyl iodide or alkyl bromide for halogen exchange and alkyl tosylate, mesylate or triflate for nucleophilic halogenation;
(c) aromatic rings activated towards electrophilic halogenation (eg. phenols) and aromatic rings activated towards nucleophilic halogenation (eg. aryl iodonium, aryl diazonium, nitroaryl).

Preferred derivatives which undergo facile alkylation are alcohols, phenols or amine groups, especially phenols and sterically-unhindered primary or secondary amines. Preferred derivatives which alkylate thiol-containing radioisotope reactants are N-haloacetyl groups, especially N-chloroacetyl and N-bromoacetyl derivatives.

The precursors may be employed under aseptic manufacture conditions to give the desired sterile, non-pyrogenic material. The precursors may also be employed under non-sterile conditions, followed by terminal sterilisation using e.g. gamma-irradiation, autoclaving, dry heat or chemical treatment (e.g. with ethylene oxide). Preferably, the precursors are employed in sterile, non-pyrogenic form. Most preferably the sterile, non-pyrogenic precursors are employed in the sealed container as described above.

The “precursor” of the kit is preferably supplied covalently attached to a solid support matrix. In that way, the desired radiopharmaceutical product forms in solution, whereas starting materials and impurities remain bound to the solid phase. Precursors for solid phase electrophilic fluorination with ¹⁸F-fluoride are described in WO 03/002489. Precursors for solid phase nucleophilic fluorination with ¹⁸F-fluoride are described in WO 03/002157. The kit may therefore contain a cartridge which can be plugged into a suitably adapted automated synthesizer. The cartridge may contain, apart from the solid support-bound precursor, a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customer requirements for radioactive concentration, volumes, time of delivery etc. Conveniently, all components of the kit are disposable to minimise the possibility of contamination between runs and will be sterile and quality assured.

In a seventh aspect, the present invention discloses the use of the imaging agent of the first embodiment for the diagnostic imaging in vivo of disease states of the mammalian body where caspase-3 is implicated. Such non-invasive imaging would relate to caspase-3 in abnormal apoptosis, and would be useful in monitoring cell death in a number of diseases. It is believed that in pathologies where cell proliferation and apoptosis is high, eg. myocardial infarction, aggressive tumours and transplant rejection, apoptosis imaging would be valuable. Such imaging would also be of value in the monitoring of chemotherapeutic drug therapy for these conditions.

In other diseases where apoptosis is thought to be important, but the number of apoptotic events is relatively rare such as in Alzheimer's disease, the available cell pool would be small and hence much more difficult to visualise. It is therefore believed likely that the apoptosis imaging agents of the present invention are best applied to pathologies where apoptosis is relatively acute, such as that seen in myocardial infarctions, aggressive tumours and transplant rejection. For those diseases in which apoptosis is more chronic, such as neuropathologies and less aggressive tumours, there may be insufficient apoptotic cells to register above background.

Essentially all treatments for cancer, including radiotherapy, chemotherapy or immunotherapy, are intended to induce apoptosis in their tumour cell targets. The imaging of apoptosis may have the capability for providing rapid, direct assessment or monitoring of the effectiveness of tumour treatment which may fundamentally alter the way cancer patients are managed. It is anticipated that patients whose tumours are responding to therapy will show significantly increased uptake of the imaging agent due to the elevated apoptotic response in the tumour. Patients whose tumours will not respond to further treatment may be identified by the failure of their tumours to increase uptake of the imaging agent post-treatment.

The evaluation of therapeutic intervention in cancer patients with measurable disease has several applications:
the evaluation of the anti-neoplastic activity of new anti-cancer drugs;
to determine efficacious therapeutic regimens;
the identification of the optimal dose and dosing schedules for new anticancer drugs;
the identification of optimal dose and dosing schedules for existing anticancer drugs and drug combinations;
the more efficient stratification of cancer patients in clinical trials into responders and non-responders of therapeutic regimens;
the efficient and timely evaluation of response of individual patients to established therapeutic anticancer regimens.

The invention is illustrated by the non-limiting Examples detailed below. Example 1 describes the synthesis of the compound 1,1,1-tris(2-aminoethyl)methane. Example 2 provides an alternative synthesis of 1,1,1-tris(2-aminoethyl)methane which avoids the use of potentially hazardous azide intermediates. Example 3 describes the synthesis of a chloronitrosoalkane precursor. Example 4 describes the synthesis of a preferred amine-substituted bifunctional diaminedioxime of the present invention (Chelator 1). Example provides the synthesis of a peptide inhibitor of the invention. Examples 6 and 8 provide the syntheses of two radiohalogenation precursors of the invention. Example 7 provides the synthesis of a non-peptide caspase-3 inhibitor of the invention. Example 9 describes a caspase-3 inhibition assay, and Example 10 a cell-based caspase-3 assay. Examples 11 and 12 provide the syntheses of suitable ¹⁸F-labelled compounds for ¹⁸F radiolabelling of caspase-3 inhibitors. Example 13 describes the radioiodination of an inhibitor of the present invention.

### EXAMPLE 1

### Synthesis of 1,1,1-tris(2-aminoethyl)methane

### (Step a): 3-(methoxycarbonylmethylene)glutaric acid dimethylester

Carbomethoxymethylenetriphenylphosphorane (167 g, 0.5 mol) in toluene (600 ml) was treated with dimethyl 3-oxoglutarate (87 g, 0.5 mol) and the reaction heated to 100° C. on an oil bath at 120° C. under an atmosphere of nitrogen for 36 h. The reaction was then concentrated in vacuo and the oily residue triturated with 40/60 petrol ether/diethylether 1:1, 600 ml. Triphenylphosphine oxide precipitated out and the supernatant liquid was decanted/filtered off. The residue on evaporation in vacuo was Kugelrohr distilled under high vacuum Bpt (oven temperature 180-200° C. at 0.2 torr) to give 3-(methoxycarbonylmethylene)glutaric acid dimethylester (89.08 g, 53%).

NMR ¹H(CDCl₃): δ 3.31 (2H, s, CH₂), 3.7(9H, s, 3×OCH₃), 3.87 (2H, s, CH₂), 5.79 (1H, s, ═CH,) ppm.

NMR ¹³C(CDCl₃), δ 36.56, CH₃, 48.7, 2×CH₃, 52.09 and 52.5 (2×CH₂); 122.3 and 146.16 C═CH; 165.9, 170.0 and 170.5 3×COO ppm.

### (Step b): Hydrogenation of 3-(methoxycarbonylmethylene)glutaric acid dimethylester

3-(methoxycarbonylmethylene)glutaric acid dimethylester (89 g, 267 mmol) in methanol (200 ml) was shaken with (10% palladium on charcoal: 50% water) (9 g) under an atmosphere of hydrogen gas (3.5 bar) for (30 h). The solution was filtered through kieselguhr and concentrated in vacuo to give 3-(methoxycarbonylmethyl)glutaric acid dimethylester as an oil, yield (84.9 g, 94%).

NMR ¹H(CDCl₃), δ 2.48 (6H, d, J=8 Hz, 3×CH₂), 2.78 (1H, hextet, J=8 Hz CH,) 3.7 (9H, s, 3×CH₃).

NMR ¹³C(CDCl₃), δ 28.6, CH; 37.50, 3×CH₃; 51.6, 3×CH₂; 172.28, 3×COO.

### (Step c): Reduction and Esterification of Trimethyl Ester to the Triacetate

Under an atmosphere of nitrogen in a 3 necked 2 L round bottomed flask lithium aluminium hydride (20 g, 588 mmol) in tetrahydrofuran (400 ml) was treated cautiously with tris(methyloxycarbonylmethyl)methane (40 g, 212 mmol) in tetrahydrofuran (200 ml) over 1 h. A strongly exothermic reaction occurred, causing the solvent to reflux strongly. The reaction was heated on an oil bath at 90° C. at reflux for 3 days. The reaction was quenched by the cautious dropwise addition of acetic acid (100 ml) until the evolution of hydrogen ceased. The stirred reaction mixture was cautiously treated with acetic anhydride solution (500 ml) at such a rate as to cause gentle reflux. The flask was equipped for distillation and stirred and then heating at 90° C. (oil bath temperature) to distil out the tetrahydrofuran. A further portion of acetic anhydride (300 ml) was added, the reaction returned to reflux configuration and stirred and heated in an oil bath at 140° C. for 5 h. The reaction was allowed to cool and filtered. The aluminium oxide precipitate was washed with ethyl acetate and the combined filtrates concentrated on a rotary evaporator at a water bath temperature of 50° C. in vacuo (5 mmHg) to afford an oil. The oil was taken up in ethyl acetate (500 ml) and washed with saturated aqueous potassium carbonate solution. The ethyl acetate solution was separated, dried over sodium sulphate, and concentrated in vacuo to afford an oil. The oil was Kugelrohr distilled in high vacuum to give tris(2-acetoxyethyl)methane (45.3 g, 96%) as an oil. Bp. 220° C. at 0.1 mmHg.

NMR ¹H(CDCl₃), δ 1.66(7H, m, 3×CH₂, CH), 2.08(1H, s, 3×CH₃); 4.1(6H, t, 3×CH₂O).

NMR ¹³C(CDCl₃), δ 20.9, CH₃; 29.34, CH; 32.17, CH₂; 62.15, CH₂O; 171, CO.

### (Step d): Removal of Acetate Groups from the Triacetate

Tris(2-acetoxyethyl)methane (45.3 g, 165 mM) in methanol (200 ml) and 880 ammonia (100 ml) was heated on an oil bath at 80° C. for 2 days. The reaction was treated with a further portion of 880 ammonia (50 ml) and heated at 80° C. in an oil bath for 24 h. A further portion of 880 ammonia (50 ml) was added and the reaction heated at 80° C. for 24 h. The reaction was then concentrated in vacuo to remove all solvents to give an oil. This was taken up into 880 ammonia (150 ml) and heated at 80° C. for 24 h. The reaction was then concentrated in vacuo to remove all solvents to give an oil. Kugelrohr distillation gave acetamide bp 170-180 0.2 mm. The bulbs containing the acetamide were washed clean and the distillation continued. Tris(2-hydroxyethyl)methane (22.53 g, 92%) distilled at bp 220° C. 0.2 mm.

NMR ¹H(CDCl₃), δ 1.45(6H, q, 3×CH₂), 2.2(1H, quintet, CH); 3.7(6H, t 3×CH₂OH); 5.5(3H, brs, 3×OH).

NMR ¹³C(CDCl₃), δ 22.13, CH; 33.95, 3×CH₂; 57.8, 3×CH₂OH.

### (Step e): Conversion of the Triol to the tris(methanesulphonate)

To an stirred ice-cooled solution of tris(2-hydroxyethyl)methane (10 g, 0.0676 mol) in dichloromethane (50 ml) was slowly dripped a solution of methanesulphonyl chloride (40 g, 0.349 mol) in dichloromethane (50 ml) under nitrogen at such a rate that the temperature did not rise above 15° C. Pyridine (21.4 g, 0.27 mol, 4 eq) dissolved in dichloromethane (50 ml) was then added drop-wise at such a rate that the temperature did not rise above 15° C., exothermic reaction. The reaction was left to stir at room temperature for 24 h and then treated with 5N hydrochloric acid solution (80 ml) and the layers separated. The aqueous layer was extracted with further dichloromethane (50 ml) and the organic extracts combined, dried over sodium sulphate, filtered and concentrated in vacuo to give tris[2-(methylsulphonyloxy)ethyl]methane contaminated with excess methanesulphonyl chloride. The theoretical yield was 25.8 g.

NMR ¹H(CDCl₃), δ 4.3 (6H, t, 2×CH₂), 3.0 (9H, s, 3×CH₃), 2 (1H, hextet, CH), 1.85 (6H, q, 3×CH₂).

### (Step f): Preparation of 1,1,1-tris(2-azidoethyl)methane

A stirred solution of tris[2-(methylsulphonyloxy)ethyl]methane [from Step 1(e), contaminated with excess methylsulphonyl chloride] (25.8 g, 67 mmol, theoretical) in dry DMF (250 ml) under nitrogen was treated with sodium azide (30.7 g, 0.47 mol) portion-wise over 15 minutes. An exotherm was observed and the reaction was cooled on an ice bath. After 30 minutes, the reaction mixture was heated on an oil bath at 50° C. for 24 h. The reaction became brown in colour. The reaction was allowed to cool, treated with dilute potassium carbonate solution (200 ml) and extracted three times with 40/60 petrol ether/diethylether 10:1 (3×150 ml). The organic extracts were washed with water (2×150 ml), dried over sodium sulphate and filtered. Ethanol (200 ml) was added to the petrol/ether solution to keep the triazide in solution and the volume reduced in vacuo to no less than 200 ml. Ethanol (200 ml) was added and reconcentrated in vacuo to remove the last traces of petrol leaving no less than 200 ml of ethanolic solution. The ethanol solution of triazide was used directly in Step 1(g).

CARE: DO NOT REMOVE ALL THE SOLVENT AS THE AZIDE IS POTENTIALLY EXPLOSIVE AND SHOULD BE KEPT IN DILUTE SOLUTION AT ALL TIMES.

Less than 0.2 ml of the solution was evaporated in vacuum to remove the ethanol and an

NMR run on this small sample:

NMR ¹H(CDCl₃), δ 3.35 (6H, t, 3×CH₂), 1.8 (1H, septet, CH,), 1.6 (6H, q, 3×CH₂).

### (Step g): Preparation of 1,1,1-tris(2-aminoethyl)methane

Tris(2-azidoethyl)methane (15.06 g, 0.0676 mol), (assuming 100% yield from previous reaction) in ethanol (200 ml) was treated with 10% palladium on charcoal (2 g, 50% water) and hydrogenated for 12 h. The reaction vessel was evacuated every 2 hours to remove nitrogen evolved from the reaction and refilled with hydrogen. A sample was taken for NMR analysis to confirm complete conversion of the triazide to the triamine.

Caution: unreduced azide could explode on distillation. The reaction was filtered through a Celite pad to remove the catalyst and concentrated in vacuo to give tris(2-aminoethyl)methane as an oil. This was further purified by Kugelrohr distillation bp. 180-200° C. at 0.4 mm/Hg to give a colourless oil (8.1 g, 82.7% overall yield from the triol).

NMR ¹H(CDCl₃), δ 2.72 (6H, t, 3×CH₂N), 1.41 (H, septet, CH), 1.39 (6H, q, 3×CH₂).

NMR ¹³C(CDCl₃), δ 39.8 (CH₂NH₂), 38.2 (CH₂.), 31.0 (CH).

### EXAMPLE 2

### Alternative Preparation of 1,1,1-tris(2-aminoethyl)methane

### (Step a): Amidation of Trimethylester with p-methoxy-benzylamine

Tris(methyloxycarbonylmethyl)methane [2 g, 8.4 mmol; prepared as in Step 1(b) above] was dissolved in p-methoxy-benzylamine (25 g, 178.6 mmol). The apparatus was set up for distillation and heated to 120° C. for 24 hrs under nitrogen flow. The progress of the reaction was monitored by the amount of methanol collected. The reaction mixture was cooled to ambient temperature and 30 ml of ethyl acetate was added, then the precipitated triamide product stirred for 30 min. The triamide was isolated by filtration and the filter cake washed several times with sufficient amounts of ethyl acetate to remove excess p-methoxy-benzylamine. After drying 4.6 g, 100%, of a white powder was obtained. The highly insoluble product was used directly in the next step without further purification or characterisation.

### (Step b): Preparation of 1,1,1-tris[2-(p-methoxybenzylamino)ethyl]methane

To a 1000 ml 3-necked round bottomed flask cooled in a ice-water bath the triamide from step 2(a) (10 g, 17.89 mmol) is carefully added to 250 ml of 1M borane solution (3.5 g, 244.3 mmol) borane. After complete addition the ice-water bath is removed and the reaction mixture slowly heated to 60° C. The reaction mixture is stirred at 60° C. for 20 hrs. A sample of the reaction mixture (1 ml) was withdrawn, and mixed with 0.5 ml 5N HCl and left standing for 30 min. To the sample 0.5 ml of 50 NaOH was added, followed by 2 ml of water and the solution was stirred until all of the white precipitate dissolved. The solution was extracted with ether (5 ml) and evaporated. The residue was dissolved in acetonitrile at a concentration of 1 mg/ml and analysed by MS. If mono- and diamide (M+H/z=520 and 534) are seen in the MS spectrum, the reaction is not complete. To complete the reaction, a further 100 ml of 1M borane THF solution is added and the reaction mixture stirred for 6 more hrs at 60° C. and a new sample withdrawn following the previous sampling procedure. Further addition of the 1M borane in THF solution is continued as necessary until there is complete conversion to the triamine.

The reaction mixture is cooled to ambient temperature and 5N HCl is slowly added, [CARE: vigorous foam formation occurs!]. HCl was added until no more gas evolution is observed. The mixture was stirred for 30 min and then evaporated. The cake was suspended in aqueous NaOH solution (20-40%; 1:2 w/v) and stirred for 30 minutes. The mixture was then diluted with water (3 volumes). The mixture was then extracted with diethylether (2×150 ml) [CARE: do not use halogenated solvents]. The combined organic phases were then washed with water (1×200 ml), brine (150 ml) and dried over magnesium sulphate. Yield after evaporation: 7.6 g, 84% as oil.

NMR ¹H(CDCl₃), δ: 1.45, (6H, m, 3×CH₂; 1.54, (1H, septet, CH); 2.60 (6H, t, 3×CH₂N); 3.68 (6H, s, ArCH₂); 3.78 (9H, s, 3×CH₃O); 6.94(6H, d, 6×Ar). 7.20(6H, d, 6×Ar).

NMR ¹³C(CDCl₃), δ: 32.17, CH; 34.44, CH₂: 47.00, CH₂: 53.56, ArCH₂: 55.25, CH—O: 113.78, Ar; 129.29, Ar; 132.61; Ar; 158.60. Ar;

### (Step c): Preparation of 1,1,1-tris(2-aminoethyl)methane

1,1,1-tris[2-(p-methoxybenzylamino)ethyl]methane (20.0 gram, 0.036 mol) was dissolved in methanol (100 ml) and Pd(OH)₂ (5.0 gram) was added. The mixture was hydrogenated (3 bar, 100° C., in an autoclave) and stirred for 5 hours. Pd(OH)₂ was added in two more portions (2×5 gram) after 10 and 15 hours respectively. The reaction mixture was filtered and the filtrate was washed with methanol. The combined organic phase was evaporated and the residue was distilled under vacuum (1×10⁻², 110° C.) to give 2.60 gram (50%) of 1,1,1-tris(2-aminoethyl)methane identical with the previously described Example 1.

### EXAMPLE 3

### Preparation of 3-chloro-3-methyl-2-nitrosobutane

A mixture of 2-methylbut-2-ene (147 ml, 1.4 mol) and isoamyl nitrite (156 ml, 1.16 mol) was cooled to −30° C. in a bath of cardice and methanol and vigorously stirred with an overhead air stirrer and treated dropwise with concentrated hydrochloric acid (140 ml, 1.68 mol) at such a rate that the temperature was maintained below −20° C. This requires about 1 h as there is a significant exotherm and care must be taken to prevent overheating. Ethanol (100 ml) was added to reduce the viscosity of the slurry that had formed at the end of the addition and the reaction stirred at −20 to −10° C. for a further 2 h to complete the reaction. The precipitate was collected by filtration under vacuum and washed with 4×30 ml of cold (−20° C.) ethanol and 100 ml of ice cold water, and dried in vacuo to give 3-chloro-3-methyl-2-nitrosobutane as a white solid. The ethanol filtrate and washings were combined and diluted with water (200 ml) and cooled and allowed to stand for 1 h at −10° C. when a further crop of 3-chloro-3-methyl-2-nitrosobutane crystallised out. The precipitate was collected by filtration and washed with the minimum of water and dried in vacuo to give a total yield of 3-chloro-3-methyl-2-nitrosobutane (115 g 0.85 mol, 73%)>98% pure by NMR.

NMR ¹H(CDCl₃), As a mixture of isomers (isomer1, 90%) 1.5 d, (2H, CH₃), 1.65 d, (4H, 2×CH₃), 5.85, q, and 5.95, q, together 1H. (isomer2, 10%), 1.76 s, (6H, 2×CH₃), 2.07(3H, CH₃).

### EXAMPLE 4

### Synthesis of bis[N-(1,1-dimethyl-2-N-hydroxyimine propyl)-2-aminoethyl]-(2-aminoethyl)methane (Chelator 1)

To a solution of tris(2-aminoethyl)methane (4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24(6H, s, 2×CH₃), 1.3(6H, s, 2×CH₃), 1.25-1.75(7H, m, 3×CH₂, CH), (3H, s, 2×CH₂), 2.58 (4H, m, CH₂N), 2.88(2H, t CH₂N₂), 5.0 (6H, s, NH₂, 2×NH, 2×OH).

NMR ¹H ((CD₃)₂SO) δ 1.1 4×CH; 1.29, 3×CH₂; 2.1 (4H, t, 2×CH₂);

NMR ¹³C((CD₃)₂SO), δ 9.0 (4×CH₃), 25.8 (2×CH₃), 31.0 2×CH₂, 34.6 CH₂, 56.8 2×CH₂N, 160.3; C═N.

HPLC conditions: flow rate 8 ml/min using a 25 mm PRP column
A = 3% ammonia solution (sp.gr = 0.88)/water; B = Acetonitrile

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### EXAMPLE 5

### Synthesis of 3-iodo-benzoyl-Asy(OMe)-Glu(OMe)-Val-Asp(OMe)-H (Compound 3)

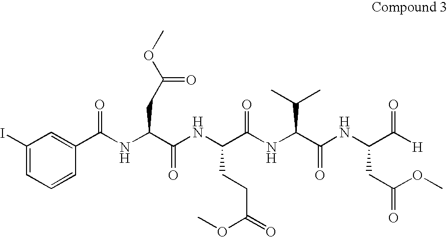

The peptidyl resin corresponding to the above sequence was assembled by standard solid-phase peptide chemistry (Barany, G; Kneib-Cordonier, N.; Mullen, D. G. (1987) Int. J. Peptide Protein Research 30, 705-739) on a H-Asp(tBu)-H NovaSyn TG resin (NovaBiochem). A manual nitrogen bubbler apparatus was used (Wellings, D. A., Atherton, E. (1997) in Methods in Enzymology (Fields, G. ed), 289, p. 53-54, Academic Press, New York). The assembled peptidyl resin 3-iodo-benzoyl-Asp(OtBu)-Glu(OtBu)-Val-Asp(OtBu)-H NovaSyn TG resin (Compound 1) was treated with trifluoroacetic acid (TFA) containing 2.5% water in order to remove the tert-butyl protecting groups. The side-chains of the aspartyl and glutamyl residues were transformed to their methyl esters using thionyl chloride (20 eq) in methanol giving peptide resin (Compound 2). The peptide product (Compound 3) was liberated from the resin by treating the peptidyl resin with 60% acetonitrile (ACN) in water containing 0.1% trifluoroacetic acid (TFA) over 4 hours. The resin residue was filtered off and the filtrate was concentrated by rotary evaporation, triturated with diethyl ether and the product was isolated by centrifugation. The product was characterized by LC-MS using an analytical RP-HPLC column (Phenomenex Luna 3μ C18(2) 50 mm×2 mm) eluted with a gradient of 0 to 70% ACN in 0.1% aq TFA over 10 min at 0.3 ml/min detecting the eluent by WV absorption at λ=214 nm and with electrospray mass spectroscopy. The desired product was confirmed at t_{R}=8.2 min with [M+H]⁺ at 733.5 m/z, expected at 733.2 m/z.

### EXAMPLE 6

### Synthesis of Trimethylstannyl Precursor for Radiohalogenation (Compound 4)

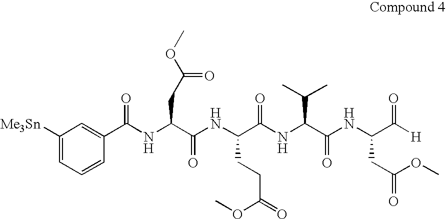

The peptide resin 3-iodo-benzoyl-Asp(OMe)-Glu(OMe)-Val-Asp(OMe)-H NovaSyn TG resin (Compound 2) from Example 5 above was stannylated using microwave technology. The 3-Iodo functionalized resin (50 mg, 0.012 mmol) was placed in an irradiation tube under argon and treated with tetrakis(triphenylphosphine)palladium (7 mg, 0.006 mmol) and hexamethylditin (7.86 mg, 5 μl, 0.024 mmol) in dry N-methylpyrrolidone (NWP) (1 ml). The tube was sealed, positioned in the cavity and irradiated for 5 minutes at 100° C. After cooling, the black coloured mixture was washed and the stannylated peptide (Compound 4) was cleaved from the resin and worked up as described in Example 5 above. The product was characterized by LC-MS using an analytical RP-HPLC column (Phenomenex Luna 3, C18(2) 50 mm×2 mm) eluted with a gradient of 10 to 80% ACN in 0.1% aq TFA over 10 min at 0.3 ml/min detecting the eluent by UV absorption at λ=214 nm and with electrospray mass spectroscopy. The desired product was confirmed at t_{R}=8.3 min with [M+H]⁺ at 771.1 m/z, expected at 771.2 m/z.

### EXAMPLE 7

### Synthesis of 1-(4-Iodobenzyl)-5-(2-methoxymethyl-pyrrolidine-1-sulfonyl)-1H-indole-2,3-dione (Compound 5)

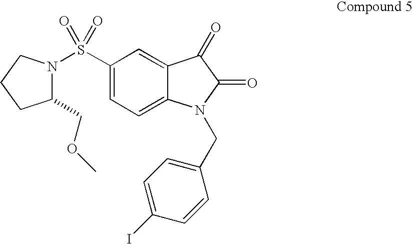

Under argon and at ambient temperature 60% sodium hydride was added to the clear and yellow solution of 5-(2-Methoxymethyl-pyrrolidine-1-sulfonyl)-1H-indole-2,3-dione (Isatin derivative; commercially available from Calbiochem Cat #218826; 50 mg, 0.154 mmol) in anhydrous DMF (5 ml). The mixture instantly turned deep purple. After stirring for 10 minutes, 4-iodobenzyl bromide (46.56 mg) in DMF (200 μl) was added and stirring at ambient temperature was continued. The purple colour faded as the reaction progressed and after 24 hours, TLC (chloroform:methanol, 8:2) rf ca 2 indicated complete reaction. DMF was then removed by evaporation under reduced pressure and the residue was flash chromatographed using chloroform:methanol (8:2) yielding 61 mg (73%) of a yellow semi-solid. The product was purified further by preparative RP-HPLC. The column (Phenomenex Luna C18 10μ, 22×250 mm) was eluted at 10 ml/min with a gradient of 30 to 80% acetonitrile (ACN) in 0.1% aq trifluoroacetic acid (TFA) over 60 min. The desired peak fractions were pooled affording pure compound 5. Analytical RP-HPLC: t_{R}=5.39 min, (Phenomenex Luna 3μ C18(2) 50 mm×2 mm, 30-80% ACN in 0.1% aq TFA over 10 min at 0.3 ml/min, λ=214 nm). Electrospray MS: [+H]⁺ of product expected at 541.0 m/z, found at 540.9 m/z.

### EXAMPLE 8

### 5-(2-Methoxymethyl-pyrrolidine-1-sulfonyl)-1-(4-trimethylstannyl-benzyl)-1H-indole-2,3-dione (Compound 6)

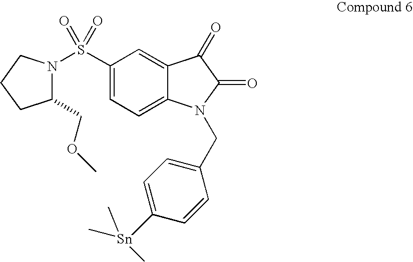

A clear yellow solution of Compound 5 (27 mg, 0.05 mmol; from Example 7 above), tetrakis(triphenylphosphine)palladium (5.78 mg, 0.005 mmol) and hexamethylditin (21 μl, 0.10 mmol) in toluene (8 ml) was heated under microwave irradiation at 120° C. for 5 minutes. The resulting black mixture was filtered. The filtrate was evaporated to dryness and the residue purified by flash chromatography using ethyl acetate:hexane (1:1) to afford the pure product as a yellow oil in 83% yield. Analytical RP-HPLC: t_{R}=7.92 min, (Phenomenex Luna 3μ C18(2) 50 mm×2 mm, 30-80% ACN in 0.1% aq TFA over 10 min at 0.3 ml/min, λ=214 nm). Electrospray MS: [+H]⁺ of product expected at 578.9 m/z, found at 578.9 m/z.

### EXAMPLE 9

### In Vitro Caspase-3 Inhibition Assay

In vitro potency of the caspase-3 inhibitors was assessed using commercially available assay kits (for example: Biomol, BIOMOL International L.P. 5120 Butler Pike, Plymouth Meeting, Pa. 19462-1202). In brief, the caspase-3 assay kit is a complete assay system designed to measure protease activity of caspase-3. It contains both a colorimetric substrate (DEVD-pNA) and a fluorogenic substrate (DEVD-AMC). Cleavage of the p-nitroanilide (pNA) from the colorimetric substrate increases absorption at 405 nm. The fluorescent assay is based on the cleavage of 7-amino-4-methylcoumarin (AMC) dye from the C-terminus of the peptide substrate. Cleavage of the dye from the substrate increases its fluorescence intensity at 460 nm. The assays are performed in a convenient 96-well microplate format. The kit is useful to screen inhibitors of caspase-3, a potential therapeutic target. An inhibitor, DEVD-CHO (aldehyde), is also included as a prototypic control inhibitor1. The DEVD amino acid sequence is derived from the caspase-3 cleavage site in PARP [poly(ADP-ribose)polymerase].

### EXAMPLE 10

### Caspase-3 Cell Assay

Jurkat and HL-60 cells were used in a cell-based model, with apoptosis induced with Staurosporin as described by Wang et al:

“A Role for Mitochondrial Bak in Apoptotic Response to Anticancer Drugs”, J. Biol. Chem., August 2001; 276: 34307-34317.

A bifunctional cell based assay is required to test caspase-3 inhibitors for their ability to enter cells and subsequently bind to the caspase-3 target. The assay is based on Fluorochrome Inhibitors of Caspases (FLICA). The inhibitors are cell permeable and once inside the cell, they bind covalently to the active caspase-3 and the FLICA fluorescence can be detected. When added to a population of cells, the FLICA probe enters each cell and covalently binds to a reactive cysteine residue that resides on the large subunit of the active caspase heterodimer, thereby inhibiting further enzymatic activity. The bound labelled reagent is retained within the cell, while any unbound reagent will diffuse out of the cell and is washed away. The green fluorescent signal is a direct measure of the amount of active caspase-3 present in the cell population at the time the reagent was added. Cells that contain the bound labelled reagent can be analyzed in 96-well plates for fluorescence.

The assay validation test articles were also caspase-3 targeted inhibitors and these were added to apoptotic cells prior to FLICA treatment. Potent test compounds would block the binding of FLICA to active caspase-3 allowing a measure of potency to be monitored by following any reduction in FLICA associated fluorescence.

### EXAMPLE 11

### Synthesis of the ¹⁸F-Labelled Derivative for N-alkylation

### Synthesis of 3-[¹⁸F]fluoropropyl tosylate

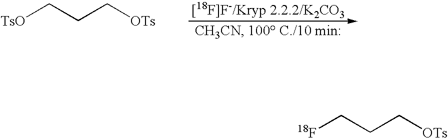

Via a two-way tap Kryptofix 222 (10 mg) in acetonitrile (300 μl) and potassium carbonate (4 mg) in water (300 μl), prepared in a glass vial, was transferred using a plastic syringe (1 ml) into a carbon glass reaction vessel sited in a brass heater. ¹⁸F-fluoride (185-370 MBq) in the target water (0.5-2 ml) was then added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and 1,3-propanediol-di-p-tosylate (5-12 mg) and acetonitrile (1 ml) was added. The pot lid was replaced and the lines capped off with stoppers. The heater was set at 100° C. and labelled at 100° C./10 mins. After labelling, 3-[¹⁸F]fluoropropyl tosylate was isolated by Gilson RP HPLC using the following conditions:

| | |
|---|---|
| Column | u-bondapak C18 7.8 × 300 mm |
| Eluent | Water (pump A):Acetonitrile (pump B) |
| Loop Size | 1 ml |
| Pump speed | 4 ml/min |
| Wavelength | 254 nm |
| Gradient | 5-90% eluent B over 20 min |
| Product Rt | 12 min |

Once isolated, the cut sample (ca. 10 ml) was diluted with water (10 ml) and loaded onto a conditioned C18 sep pak. The sep pak was dried with nitrogen for 15 mins and flushed off with an organic solvent, pyridine (2 ml), acetonitrile (2 ml) or DMF (2 ml). Approx. 99% of the activity was flushed off.

3-[¹⁸F]fluoropropyl tosylate is used to N-alkylate amines by refluxing in pyridine.

### EXAMPLE 12

### [¹⁸F]-Thiol Derivative for S-alkylation

### Step (a): Preparation of 3-[¹⁸F]fluoro-tritylsulfanyl-propane

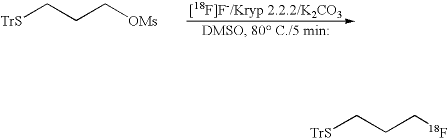

Via a two-way tap Kryptofix 222 (10 mg) in acetonitrile (800 μl) and potassium carbonate (1 mg) in water (50 μl), prepared in a glass vial, was transferred using a plastic syringe (1 ml) to the carbon glass reaction vessel situated in the brass heater. ¹⁸F-fluoride (185-370 MBq) in the target water (0.5-2 ml) was then also added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and trimethyl-(3-tritylsulfanyl-propoxy)silane (1-2 mg) and DMSO (0.2 ml) was added. The pot lid was replaced and the lines capped off with stoppers. The heater was set at 80° C. and labelled at 80° C./5 mins. After labelling, the reaction mixture was analysed by RP HPLC using the following HPLC conditions:

| Column | u-bondapak C18 7.8 × 300 mm | |
|---|---|---|
| Eluent | 0.1% TFA/Water (pump A):0.1% | |
| | TFA/Acetonitrile (pump B) | |
| Loop Size | 100 ul | |
| Pump speed | 4 ml/min | |
| Wavelength | 254 nm | |
| Gradient | 1 mins | 40% B |
| | 15 mins | 40-80% B |
| | 5 mins | 80% B |

The reaction mixture was diluted with DMSO/water (1:1 v/v, 0.15 ml) and loaded onto a conditioned t-C18 sep-pak. The cartridge was washed with water (10 ml), dried with nitrogen and 3-[¹⁸F]fluoro-1-tritylsulfanyl-propane was eluted with 4 aliquots of acetonitrile (0.5 ml per aliquot).

### Step (b): Preparation of 3-[¹⁸F]fluoro-propane-1-thiol

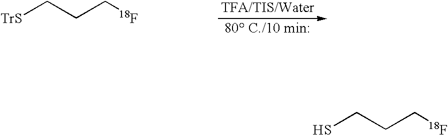

A solution of 3-[¹⁸F]fluoro-1-tritylsulfanyl-propane in acetonitrile (1-2 ml) was evaporated to dryness using a stream of nitrogen at 100° C./10 mins. A mixture of TFA (0.05 ml), triisopropylsilane (0.01 ml) and water (0.01 ml) was added followed by heating at 80° C./10 mins to produce 3-[¹⁸F]fluoro-propane-1-thiol.

### Step (c): Reaction with —N(CO)CH₂Cl Precursors

A general procedure for labelling a chloroacetyl precursor is to cool the reaction vessel containing the 3-[¹⁸F]fluoro-1-mercapto-propane from Step (b) with compressed air, and then to add ammonia (27% in water, 0.1 ml) and the precursor (1 mg) in water (0.05 ml). The mixture is heated at 80° C./10 mins.

### EXAMPLE 13

### [¹²³I]-Radiolabelling of a Caspase-3 Inhibitor

### Step (a): Alternative Synthesis of Compound 5

Compound 5 is the non-radioactive analogue, ie. where the iodine isotope is ¹²⁷I, and was prepared according to Scheme 4 below.

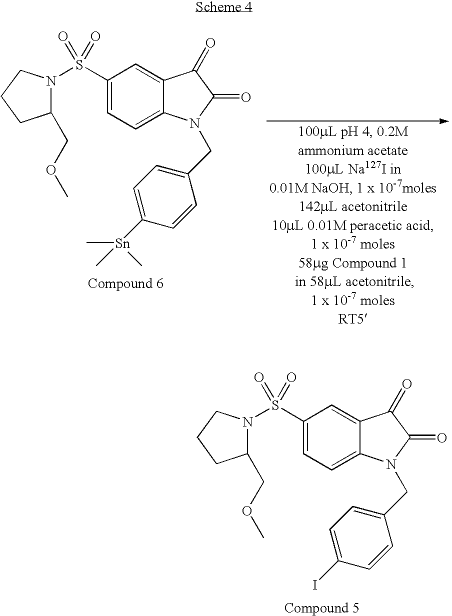

Mass spectroscopic analysis of confirmed the identity of Compound 5.

### Step (b): Synthesis of Compound 5A

For the preparation of the ¹²³I-labelled Compound 5 (Compound 5A), a protocol similar to step (a) was followed. To between 8-30 μl of carrier-free sodium [¹²³I]iodide, was added 100 μl pH 4, 0.2M ammonium acetate buffer, 10 μl sodium [¹²⁷I]iodide, 15 mg/100 ml solution sodium iodide in 0.01M sodium hydroxide, (1×10⁻⁸ moles) and 50 μl acetonitrile. The reagents were mixed and transferred to a silanised P15 vial. Finally 10 μl 0.001M peracetic acid solution (1×10⁻⁸ moles) and 58 μl of a 1 mg/ml solution of Compound 6 in acetonitrile (1×10⁻⁷ moles) were added. [¹²³I]-Compound 5 (Compound 5A) was HPLC purified and diluted in pH 7.4, 50 mM sodium phosphate buffer with 10% ethanol (to aid solubility) to 20 MBq/ml or 100 MBq/ml with a typical specific activity of 14 MBq/nmole and 41 MBq/nmole respectively. Both preparations were found to be stable at pH 7.5 (>95% RCP over 4 hours). Co-elution with the ¹²⁷I standard from step (a) was observed confirming identity.

What is claimed is:

1. An imaging agent which comprises a synthetic caspase-3 inhibitor labelled with an imaging moiety, wherein the caspase-3 inhibitor has a Kᵢ for caspase-3 of less than 2000 nM, and wherein following administration of said labelled caspase-3 inhibitor to the mammalian body in vivo, the imaging moiety can be detected either externally in a non-invasive manner or via use of detectors designed for use in vivo

2. (canceled)

3. The imaging agent of claim 1, where the synthetic caspase-3 inhibitor has a molecular weight of 150 to 3000 Daltons.

4. The imaging agent of claim 1, where the imaging moiety comprises:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) an optical dye suitable for in vivo imaging.

5. The imaging agent of claim 1, which further comprises a 4 to 20-mer leader peptide sequence, wherein said leader peptide facilitates cell membrane transport from the outside to the inside of a mammalian cell in vivo.

6. The imaging agent of claim 5 where the synthetic caspase-3 inhibitor conjugate is of Formula I: 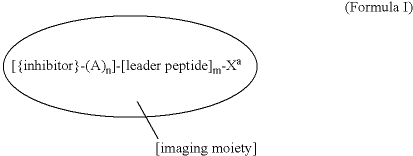
where:
{inhibitor} is a caspase-3 inhibitor with a Ki for caspase-3 of less than 2000 nM;
[leader peptide] is as defined in claim 5 and is attached by either its' amine or carboxyl terminus;
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid or a monodisperse polyethyleneglycol (PEG) building block;
R is independently chosen from H, C₁₋₄ alkyl, C₂₋₄ alkenyl, C₂₋₄ alkynyl, C₁₋₄ alkoxyalkyl or C₁₋₄ hydroxyalkyl;
n is an integer of value 0 to 10,
m is 0 or 1;
and X^{a} is H, OH, Hal, NH₂, C₁₋₄ alkyl, C₁₋₄ alkoxy, C₁₋₄ alkoxyalkyl, C₁₋₄ hydroxyalkyl or X^{a} is the imaging moiety.

7. The imaging agent of claim 1, where the radioactive metal ion is a gamma emitter or a positron emitter.

8. The imaging agent of claim 7, where the radioactive metal ion is ^{99m}Tc, ¹¹¹In, ⁶⁴Cu, ⁶⁷Cu, ⁶⁷Ga or ⁶⁸Ga.

9. The imaging agent of claim 1, where the paramagnetic metal ion is Gd(III), Mn(II) or Fe(III).

10. The imaging agent of claim 1, where the gamma-emitting radioactive halogen is ¹²³I.

11. The imaging agent of claim 1, where the positron-emitting radioactive non-metal is chosen from ¹⁸F, ¹¹C, ¹²⁴I or ¹³N.

12. The imaging agent of claim 1, where the synthetic caspase-3 inhibitor comprises one or more of the caspase-3 inhibitors defined in (i) to (ix):
(i) a tetrapeptide derivative of Formula III
Z¹-Asp-Xaa1-Xaa2-Asp-X¹ (III)
where Z¹ is a metabolism inhibiting group attached to the N-terminus of the tetrapeptide;
Xaa1 and Xaa2 are independently any amino acid;
X¹ is an —R¹ or —CH₂OR² group attached to the carboxy terminus of the tetrapeptide;
where R¹ is H, —CH₂F, —CH₂Cl, C₁₋₅ alkyl, C₁₋₅ alkoxy or —(CH₂)_{q}Ar¹, where q is an integer of value 1 to 6 and Ar¹ is C₆₋₁₂ aryl, C₅₋₁₂ alkyl-aryl, C₅₋₁₂ fluoro-substituted aryl, or C₃₋₁₂ heteroaryl;
R² is C₁₋₅ alkyl, C₁₋₁₀ acyl or Ar¹;
(ii) a quinazoline or anilinoquinazoline;
(iii) a 2-oxindole sulphonamide;
(iv) an oxoazepinoindoline;
(v) a compound of Formula IV 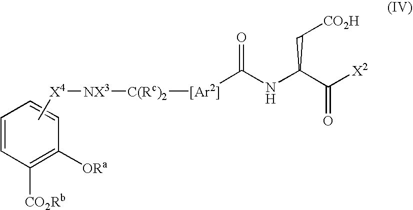
where X² is H, C₁₋₅ alkyl or —(CH₂)ᵣ—(S)ₛ(CH₂)ₜAr³, where r and t are integers of value 0 to 6, s is 0 or 1 and Ar³ is C₆₋₁₂ aryl, C₅₋₁₂ alkyl-substituted aryl, C₅₋₁₂ halo-substituted aryl, or C₃₋₁₂ heteroaryl;
Ar² is C₆₋₁₂ aryl or C₃₋₁₂ heteroaryl;
X³ is an R^{b} group;
X⁴ —SO₂— or —CR₂—
R^{a} is H, C₁₋₅ alkyl or p^{GP} where p^{GP} is a protecting group;
R^{b} is an R^{a} group or C₁₋₅ acyl;
each R^{c} is independently H or C₁₋₅ alkyl;
(vi) a compound of Formula V 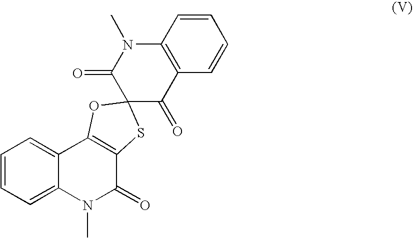
(vii) a pyrazinone;
(viii) a dipeptide of Formula VI:
Z¹-Val-Asp-CH₂—S—R¹ (VI)
where the —CH₂SR¹ group is attached to the carboxy terminus of the dipeptides, and Z¹ and R¹ are as defined for Formula (III);
(ix) a salicylic acid sulphonamide of Formula XI: 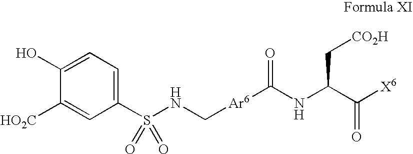
Where Ar⁶ is a 5 or 6-membered C₄₋₆ aryl or heteroaryl ring, and X6 is H or —CH₂SR², where R2 is as defined above.

13. The imaging agent of claim 12, where the synthetic caspase-3 inhibitor comprises:
(i) a tetrapeptide of Formula III; or
(ii) a 2-oxindole sulphonamide; or
(iii) a dipeptide of Formula VI.

14. The imaging agent of claim 1, where the synthetic caspase-3 inhibitor is selective for caspase-3 over caspase-1, by a factor of at least 50.

15. The imaging agent of claim 13, where the synthetic caspase-3 inhibitor comprises a tetrapeptide of Formula III or a dipeptide of Formula VI.

16. A pharmaceutical composition which comprises the imaging agent of claim 1 together with a biocompatible carrier, in a form suitable for mammalian administration.

17. A radiopharmaceutical composition which comprises the imaging agent of claim 1 wherein the imaging moiety is radioactive, together with a biocompatible carrier, in a form suitable for mammalian administration.

18. The radiopharmaceutical composition of claim 17, where the imaging moiety comprises a positron-emitting radioactive non-metal or a gamma-emitting radioactive halogen.

19. The radiopharmaceutical composition of claim 17, where the imaging moiety comprises a radioactive metal ion.

20. A conjugate of a synthetic caspase-3 inhibitor with a ligand, wherein the caspase-3 inhibitor has a Kᵢ for caspase-3 of less than 500 nM, and wherein said ligand is capable of forming a metal complex with a radioactive or paramagnetic metal ion.

21. The conjugate of claim 20, of Formula Ib: 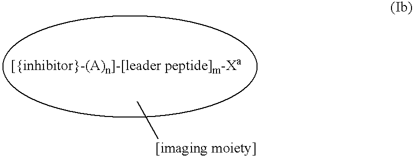
where
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid or a monodisperse polyethyleneglycol (PEG) building block;
R is independently chosen from H, C₁₋₄ alkyl, C₂₋₄ alkenyl, C₂₋₄ alkynyl, C₁₋₄ alkoxyalkyl or C₄ hydroxyalkyl,
n is an integer of value 0 to 10,
m is or 1
and X^{a} is H, OH, Hal, NH₂, C₁₋₄ alkyl, C₁₋₄ alkoxy, C₁₋₄ alkoxyalkyl, C₁₋₄ hydroxyalkyl or X^{a} is the imaging moiety.

22. The conjugate of claim 20, wherein the ligand is a chelating agent.

23. The conjugate of claim 22, wherein the chelating agent has a diaminedioxime, N₂S₂, or N₃S donor set.

24. A kit for the preparation of the radiopharmaceutical composition of claim 19, which comprises the conjugate of a synthetic caspase-3 inhibitor with a ligand, wherein the caspase-3 inhibitor has a Kᵢ for caspase-3 of less than 500 nM, and wherein said ligand is capable of forming a metal complex with a radioactive or paramagnetic metal ion.

25. The kit of claim 24, where the radioactive metal ion is ^{99m}Tc, and the kit further comprises a biocompatible reductant.

26. A kit for the preparation of the radiopharmaceutical composition of claim 18, which comprises a precursor, said precursor being a non-radioactive derivative of a caspase-3 inhibitor, wherein the caspase-3 inhibitor has a Kᵢ for caspase-3 of less than 2000 nM, wherein said non-radioactive derivative is capable of reaction with a source of the positron-emitting radioactive non-metal or gamma-emitting radioactive halogen to give the desired radiopharmaceutical.

27. The kit of claim 26 where the precursor is in sterile, apyrogenic form.

28. The kit of claim 26, where the source of the positron-emitting radioactive non-metal or gamma-emitting radioactive halogen is chosen from:
a a halide ion or F⁺ or I⁺; or
b an alkylating agent chosen from an alkyl or fluoroalkyl halide, tosylate, triflate or mesylate;

29. The kit of claim 26, where the non-radioactive derivative is chosen from:
a an organometallic derivative such as a trialkylstannane or a trialkylsilane;
b a derivative containing an alkyl halide, alkyl tosylate or alkyl mesylate for nucleophilic substitution;
c a derivative containing an aromatic ring activated towards nucleophilic or electrophilic substitution;
d a derivative containing a functional group which undergoes facile alkylation;
e a derivative which alkylates thiol-containing compounds to give a thioether-containing product.

30. The kit of claim 26, where the precursor is bound to a solid phase.

31. Use of the imaging agent of claim 1 in a method of diagnosis of a caspase-3 implicated disease state of the mammalian body, wherein said mammal is previously administered with the pharmaceutical composition which comprises the imaging agent of claim 1 together with a biocompatible carrier, in a form suitable for mammalian administration, or the radiopharmaceutical composition which comprises the imaging agent of claim 1 wherein the imaging moiety is radioactive, together with a biocompatible carrier, in a form suitable for mammalian administration.
